(19) United States
(12) Patent Application Publication
(10) Pub. No.: US 2022/0095930 A1
(43) Pub. Date: Mar. 31, 2022

(54) SYSTEMS, DEVICES, AND METHODS FOR NON-INVASIVE CARDIAC MONITORING

(71) Applicant: VivaLNK, Inc., Campbell, CA (US)

(72) Inventors: Jiang LI, Cupertino, CA (US); Troy Peterson Kling, San Jose, CA (US); Niel Starksen, Los Altos, CA (US)

(21) Appl. No.: 17/491,079

(22) Filed: Sep. 30, 2021

Related U.S. Application Data
(63) Continuation of application No. PCT/US2020/026200, filed on Apr. 1, 2020.
(60) Provisional application No. 62/827,726, filed on Apr. 1, 2019.

(51) Int. Cl. A61B 5/022; A61B 5/352; A61B 5/28; A61B 5/00

(52) U.S. Cl. CPC A61B 5/022; A61B 5/352; A61B 2562/0219; A61B 5/6823; A61B 5/28

(57) ABSTRACT

Devices, systems, and methods herein relate to non-invasive cardiac monitoring of a cardiac parameter such as blood pressure. These systems and methods may receive and process user and cohort cardiac data to generate mechanical cardiac parameter values used to estimate blood pressure. In some variations, a method may include the steps of receiving mechanical cardiac data of a user measured using an accelerometer. A mechanical cardiac parameter value for a first time period and a second time period may be generated from the mechanical cardiac data. The blood pressure of the user may be estimated based on a change in the mechanical cardiac parameter value between the first and second time periods.

### CROSS-REFERENCE TO RELATED APPLICATIONS

This application is a continuation of International Application No. PCT/US2020/026200, filed on Apr. 1, 2020, which claims priority to U.S. Provisional Application Ser. No. 62/827,726, filed on Apr. 1, 2019, the content of each of which is hereby incorporated by reference in its entirety.

### TECHNICAL FIELD

Devices, systems, and methods herein relate to non-invasive cardiac monitoring to estimate a physiological characteristic of a patient.

### BACKGROUND

Vital signs such as blood pressure and heart rate are commonly used to indicate the status and health of a subject. Measurement of blood pressure is commonly performed in a clinical setting using a sphygmomanometer and pressure cuff, which may be cumbersome and impractical for continuous and/or ambulatory blood pressure monitoring. Other blood pressure measurement techniques include calculations based on pulse transit time (PTT). PTT measures the time it takes a pulse wave to travel between two arterial sites using electrocardiogram (ECG) and photoplethysmogram (PPG) measurements. However, the accuracy of blood pressure estimates using PTT may be adversely affected by factors such as a subject's age, gender, body composition, strength level, weight, anatomical variation, and the like. Furthermore, blood pressure estimates relying on PTT using a timepoint indicated by an ECG R-wave may be inaccurate due to the inclusion of a pre-ejection period (PEP), during which the left ventricle of the heart undergoes isovolumic contraction and where the blood has not yet left the chamber via the aortic valve. A more accurate estimate of the blood pulse wave traveling from the heart to a distal location may rely on mechanical indicators of the valve opening. As such, additional devices, systems, and methods for blood pressure estimation may be desirable.

### SUMMARY

Described here are cardiac monitoring devices, systems, and methods for providing real-time, non-invasive monitoring of one or more cardiac parameters, which may be used to estimate a physiological characteristic of a patient, such as blood pressure. These systems and methods may, for example, receive cardiac data of a user (e.g., reference blood pressure, ECG data, seismocardiogram (SCG) data), process the data to generate values for at least one mechanical cardiac parameter, and estimate a physiological characteristic of interest, such as blood pressure, based on the received and processed data. This may, for example, allow insight into a vital sign of a user on a continuous or semi-continuous, real-time basis. The devices described herein for use in estimating the physiological characteristic may be compact and portable such that they may allow for continuous or semi-continuous, real-time monitoring without restricting day-to-day activities of the user.

In some variations, a method of estimating blood pressure may comprise receiving mechanical cardiac data of a user measured using an accelerometer, generating a mechanical cardiac parameter value for a first time period and a second time period from the mechanical cardiac data, and estimating the blood pressure of the user based on a change in the mechanical cardiac parameter value between the first and second time periods. In some variations, the method may further comprise receiving a reference blood pressure of a user and cohort cardiac data associated with the user. In some of these variations, the estimated blood pressure may also be based on the reference blood pressure and the cohort cardiac data.

In some variations, generating the mechanical cardiac parameter value may comprise generating mechanical cardiac parameter values for a plurality of mechanical cardiac parameters. In these variations, the estimated blood pressure may be based on a sum of the changes in the mechanical cardiac parameter values between the first and second time periods for the plurality of mechanical cardiac parameters.

In some of these variations, at least one of the plurality of mechanical cardiac parameters may be selected from the group consisting of an SCG wave amplitude, a maximal L2 distance, an area under a power spectral density curve, a sample entropy, and an R wave wavelength. In some variations, the mechanical cardiac data may comprise an SCG wave. In some of these variations, the SCG wave may comprise an SCG1 wave and an SCG2 wave.

In some variations, the mechanical cardiac data may comprise a plurality of SCG waves for each of the first time period and the second time period. Generating mechanical cardiac parameter values may comprise generating an average SCG wave for the first time period and the second time period. In some of these variations, the mechanical cardiac parameter values may be derived from the average SCG waves. In some of these variations, the mechanical cardiac parameter may comprise an SCG wave amplitude, a maximal L2 norm or distance, an area under a power spectral density curve, a sample entropy, or an R wave wavelength. In some variations, the accelerometer may be a seismocardiogram (SCG) sensor.

In some variations, the method may further comprise receiving electrical cardiac data measured using an electrode. In some of these variations, the electrode may be an electrocardiogram (ECG) electrode and the accelerometer may be a seismocardiogram (SCG) sensor. In some of these variations, the method may further comprise measuring the electrical cardiac data as an ECG signal and the mechanical cardiac data as an SCG signal.

In some of these variations, the method may further comprise generating a plurality of electrical cardiac parameter values from the electrical cardiac data. The electrical cardiac parameter may comprise one or more of a heart rate, an R wave timepoint, and a T wave timepoint. In some of these variations, generating the plurality of electrical cardiac parameter values may comprise generating the R wave timepoint on an ECG waveform using sliding window integration. In some of these variations, generating the plurality of electrical cardiac parameter values may comprise generating the T wave timepoint on an ECG waveform using the R wave timepoint and a derivative of the electrical cardiac data. In some of these variations, the method may further comprise generating an SCG wave timepoint from the mechanical cardiac data using the R wave and T wave timepoint.

In some variations, the mechanical cardiac data may comprise first, second, and third seismocardiograph waveforms measured along respective axes. The method may further comprise generating a fourth seismocardiograph waveform comprising the first, second, and third seismocardiograph waveforms.

In some variations, estimating the blood pressure is given by: BPₑₛₜ=BP_{ref}+Σᵢ₌₁ⁿβᵢ(x_{2,i}−x_{1,i}), where i is a number of mechanical cardiac parameters, BPₑₛₜ is the estimated blood pressure of the user, BP_{ref} is the reference blood pressure of the user, βᵢ is an i^{th} cohort mechanical cardiac parameter value, x_{1,i} is the first value of the i^{th} mechanical cardiac parameter, and x_{2,i} is the second value of the i^{th} mechanical cardiac parameter.

In some variations, the accelerometer may be releasably coupled to skin of the user over a left ribcage. In some variations, the accelerometer may be coupled to the user perpendicular to a mid-clavicular line and proximate to the intersection of a fifth intercostal space of the ribcage and the mid-clavicular line.

In some variations, the cohort cardiac data may be grouped by one or more of age, gender, race, and body mass index. In some variations, the estimated blood pressure comprises one or more of systolic blood pressure and diastolic blood pressure. In some variations, the first time period may be a reference time period. The mechanical cardiac data may be initially measured using the accelerometer during the reference time period.

In some variations, a method of estimating blood pressure may include the steps of receiving a reference blood pressure of a user, cohort cardiac data associated with the user, and cardiac data of the user at first and second time periods. The method may further comprise measuring the cardiac data using an electrode and an accelerometer. The cardiac data may be processed to generate first and second values for a mechanical cardiac parameter corresponding to the respective first and second time periods. The method may further comprise estimating the blood pressure of the user based on the reference blood pressure, the cohort cardiac data, and a change between the first and second values for the mechanical cardiac parameter.

In some variations, a method of estimating blood pressure may include the steps of receiving a reference blood pressure of a user and cardiac data of the user at first and second time periods. The method may further comprise measuring the cardiac data using an electrocardiogram (ECG) sensor and a seismocardiogram (SCG) sensor each attached on the skin of the user's left chest. The method may further comprise processing the cardiac data to generate electrical cardiac parameter values corresponding to an R wave timepoint and a T wave timepoint, and to generate first and second values for a mechanical cardiac parameter corresponding to the respective first and second time periods based at least in part on the R wave and T wave timepoints. The method may further comprise receiving cohort cardiac data associated with the user for the mechanical cardiac parameter. The method may further comprise estimating the blood pressure of the user based on the reference blood pressure, the cohort cardiac data, and a change between the first and second values for the mechanical cardiac parameter.

Also described here are systems. In some variations, a cardiac monitoring system may comprise a cardiac monitor comprising a cardiac sensor comprising an accelerometer. The cardiac sensor may be configured to releasably attach to skin of the user's left chest and to measure cardiac data at first and second time periods. The cardiac monitor may further comprise a communication device configured to establish a communication channel A non-transitory processor-readable storage medium may be configured to be executed by a processor and comprise instructions to receive the cardiac data using the communication channel The instructions may further comprise generating a mechanical cardiac parameter value from the cardiac data for a first time period and a second time period. The instructions may further comprise estimating a blood pressure of the user based on a change in the mechanical cardiac parameter values between the first and second time periods.

In some variations, the non-transitory processor-readable storage medium further comprises instructions to retrieve a reference blood pressure of the user and cohort cardiac data associated with the user. In some variations, the estimated blood pressure may also be based on the reference blood pressure and the cohort cardiac data.

In some variations, the instructions to generate a mechanical cardiac parameter value may comprise instructions to generate mechanical cardiac parameter values for a plurality of mechanical cardiac parameters. The estimated blood pressure may be based on a sum of the changes in the mechanical cardiac parameter values between the first and second time periods for the plurality of mechanical cardiac parameters.

In some variations, the mechanical cardiac parameter may comprise an SCG wave amplitude, a maximal L2 norm, an area under a power spectral density curve, a sample entropy, and an R wave wavelength. In some variations, the cardiac monitor may further comprise an electrode configured to measure cardiac data associated with electrical activity of a heart. In some of these variations, the electrode may be an electrocardiogram (ECG) electrode and the accelerometer may be a seismocardiogram (SCG) sensor.

The devices and systems described herein may execute one or more steps of the methods described in more detail herein. In some variations, a non-transitory processor-readable storage medium may be configured to be executed by a processor and may comprise instructions to receive cardiac data of a user for a first time period and a second time period. The instructions may further comprise retrieving a reference blood pressure of the user and cohort cardiac data associated with the user. The instructions may further comprise processing the cardiac data to generate first and second values for a mechanical cardiac parameter corresponding to the respective first and second time periods. The instructions may further comprise estimating a blood pressure of the user based on the reference blood pressure, the cohort cardiac data, and a change between the first and second values for the mechanical cardiac parameter.

Also described here are devices. In some variations, a device for estimating blood may comprise a transceiver configured to receive one or more of a reference blood pressure of a user, cohort cardiac data associated with the user, cardiac data at first and second time periods, and first and second values for a mechanical cardiac parameter corresponding to the respective first and second time periods. A processor may be configured to estimate the blood pressure of a user based on the reference blood pressure, the cohort cardiac data, and a change between the first and second values for the mechanical cardiac parameter.

In some variations, a cardiac monitor may be provided, and may include a cardiac sensor comprising an electrode and an accelerometer. The cardiac sensor may be configured to releasably attach to skin on the user's left chest and to measure cardiac data at a first time period and a second time period. A memory may be configured to store the measured cardiac data, a reference blood pressure of the user, and cohort cardiac data associated with the user. A processor may be configured to process the cardiac data to generate first and second values for a mechanical cardiac parameter corresponding to the respective first and second time periods. The processor may further be configured to estimate the blood pressure of the user based on the reference blood pressure, the cohort cardiac data, and a change between the first and second values for the mechanical cardiac parameter.

### BRIEF DESCRIPTION OF THE DRAWINGS

FIGS. 1A and 1B are top and bottom perspective views of an illustrative variation of a cardiac monitor that may be used to estimate blood pressure. FIG. 1C is a side view of the cardiac monitor shown in FIG. 1A. FIG. 1D is a cross-sectional side view of the cardiac monitor shown in FIG. 1A.

FIG. 2 is an exploded perspective view of an illustrative variation of a cardiac monitor.

FIG. 3A is a plan view of an illustrative variation of an adhesive portion for use with the cardiac monitor. FIG. 3B is an exploded perspective view of the adhesive portion shown in FIG. 3A.

FIG. 4 is a schematic view of an illustrative variation of a cardiac monitor attached to a user.

FIG. 5 is a schematic block diagram of an illustrative variation of a cardiac monitoring system.

FIG. 6A is a perspective view of an illustrative variation of a dock. FIG. 6B is a cross-sectional side view of the dock shown in FIG. 6A.

FIG. 7 is a flowchart describing an illustrative variation of a method of estimating blood pressure.

FIGS. 8A and 8B are illustrative variations of electrocardiogram (ECG) waveforms of electrical cardiac activity and seismocardiogram (SCG) waveforms of mechanical cardiac activity.

FIG. 9 is a flowchart describing an illustrative variation of a method of determining electrical cardiac parameter values including R wave and T wave timepoints.

FIG. 10 is a flowchart describing an illustrative variation of a method of determining mechanical cardiac parameter values.

FIG. 11 is a flowchart describing an illustrative variation of a method of determining mechanical cardiac parameter values.

FIG. 12 is a flowchart describing an illustrative variation of a method of estimating blood pressure.

FIG. 13 is a graph of measured and estimated blood pressure data for males over 70 years of age.

FIG. 14 is a graph of measured and estimated blood pressure data for females between 20 and 29 years of age.

FIGS. 15A-15D are histograms of demographic information for a set of test subjects. FIG. 15A is an age distribution graph. FIG. 15B is a body mass index (BMI) distribution graph. FIG. 15C is a resting systolic blood pressure graph. FIG. 15D is a resting diastolic blood pressure graph.

FIG. 16A is a plot of systolic blood pressure error. FIG. 16B is a plot of diastolic blood pressure error.

### DETAILED DESCRIPTION

Described here are systems, devices, and methods for non-invasively monitoring a physiological characteristic of a patient, such as blood pressure. These systems, devices, and methods may receive and process user cardiac data to generate mechanical cardiac parameter values, which may be used to estimate blood pressure. The systems, devices, and methods may further receive a reference blood pressure and cohort data, which may be used in conjunction with the mechanical cardiac parameters to estimate blood pressure. In some variations, user cardiac data may be continuously measured non-invasively using a removably attachable cardiac monitor. As used herein, cardiac data may refer to electrical and/or mechanical cardiac data measured over a predetermined time period.

Generally, the systems described here may comprise a cardiac monitor and one or more of a computing device, a dock, a network, a server, and a database. The cardiac monitor may measure cardiac data of a user, and may, in some variations, transmit the cardiac data to a computing device, a dock, a remote server, and/or a database for processing and analysis. In other variations, the cardiac data may be processed and analyzed on the cardiac monitor itself. As mentioned above, the cardiac data may comprise electrical cardiac data and/or mechanical cardiac data. The electrical cardiac data may be measured using a plurality of electrodes and the mechanical cardiac data may be measured using an accelerometer. The cardiac data may be processed and analyzed to generate cardiac parameters, for example, mechanical cardiac parameters and electrical cardiac parameters. In some variations, electrical cardiac parameter values such as ECG R wave timepoints and T wave timepoints may be used to determine mechanical cardiac parameter values such as SCG wave timepoints. Then, only values of one or more mechanical cardiac parameters may be used to estimate a physiological characteristic of a user, for example, blood pressure (e.g., systolic and diastolic blood pressure). For example, the physiological characteristic may be estimated solely based on the mechanical cardiac parameter values generated from the mechanical cardiac data. Put another way, the physiological characteristic may be estimated without using values from electrical cardiac data directly.

The measurement of cardiac data and estimation of a user physiological characteristic may be performed for predetermined intervals or continuously. The results of the estimation may be output to one or more of the cardiac monitor, computing device, dock, network, server, database, combinations thereof, and the like. Additionally (e.g., concurrently) or alternatively, the resultant blood pressure estimation may be output to one or more of a health care professional and designated users (e.g., partner, family, support group).

### I. Systems

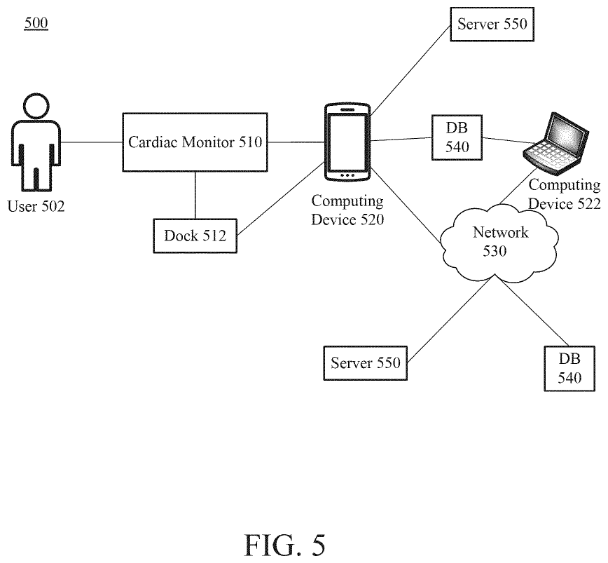

A cardiac monitoring system may include one or more of the components necessary to measure and/or generate a cardiac parameter value using the devices as described herein. FIG. 5 is a block diagram of a variation of a cardiac monitoring system (500). As shown there, the system (500) may comprise a cardiac monitor (510), a dock (512), a computing device (520, 522), a network (530), a database (540), and a server (550). The system (500) may comprise a cardiac monitor (510) configured to removably attach to a user (502) and to measure cardiac data of the user. As described in more detail herein, the cardiac monitor (510) may comprise a cardiac sensor comprising a plurality of electrodes (e.g., a pair of electrodes) and an accelerometer. In some variations, the cardiac monitor (510) may be configured to releasably attach to the skin of a user's left chest (for example, over the user's ribcage or near and below the user's clavicle).

The cardiac monitor (510) may further comprise a communication device configured to establish a communication channel. The cardiac monitor (510) may be coupled to a computing device (520) through one or more wired or wireless communication channels. The computing device (520) may be operatively coupled one or more networks (530), databases (540), and/or servers (550). The network (530) may comprise one or more databases (540) and servers (550). In some variations, another user (not shown) such as a health care professional may be allowed access to the cardiac monitor (510) through a respective computing device (522). In some variations, the cardiac monitor (510) may be coupled directly to any of the network (530), the database (540), and the server (550). In some variations, the cardiac monitor (510) may be coupled to a dock (512) for storage, to recharge, to transfer data, combinations thereof, and the like.

In some variations, the measured cardiac data may be processed on any one of the devices of the system (500), while in other variations, the processing may be distributed throughout a plurality of devices. In some variations, cardiac data processing may comprise filtering data (e.g., reducing noise, averaging waveforms), determining key events in a cardiac waveform (e.g., R wave and T wave in an ECG waveform, SCG1 and SCG2 waves in SCG waveform), and generating mechanical cardiac parameter values used to estimate a physiological characteristic. In some variations, cardiac data and user identifying information may be encrypted and stored according to HIPAA regulations.

### Cardiac Monitor

Generally, the cardiac monitors described here may be configured to measure user cardiac data during a plurality of time periods. In some variations, the measured cardiac data may be transmitted to a computing device for data processing and blood pressure estimation as described herein. The cardiac monitor may be controlled from one or more computing devices. In some variations, the cardiac monitors described here may be configured to perform only a subset of the measurement, processing, and estimation steps described herein.

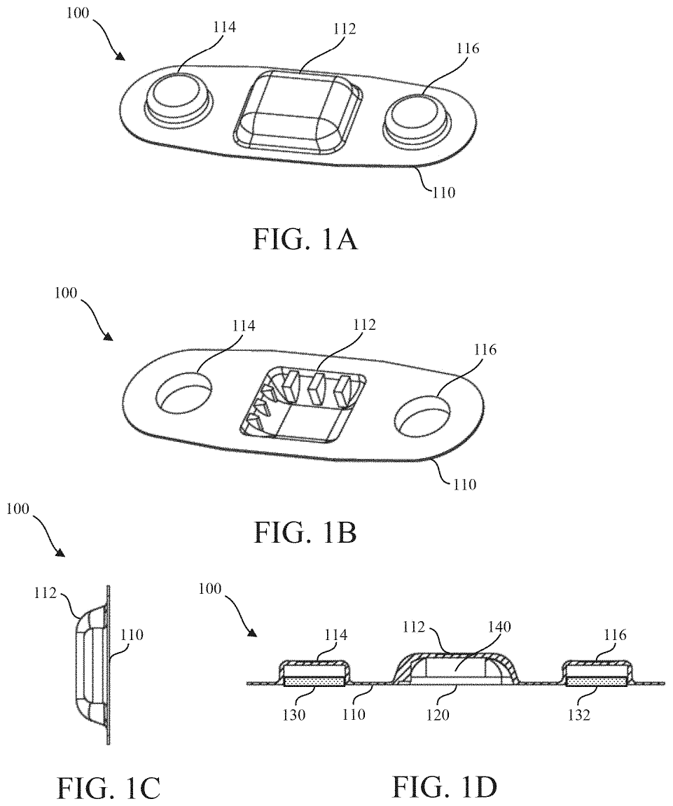

FIGS. 1A and 1B are top and bottom perspective views of a variation of a cardiac monitor (100). FIG. 1C is a side view, and FIG. 1D is a cross-sectional side view, of the cardiac monitor (100) shown in FIG. 1A. As depicted there, the cardiac monitor (100) may comprise a substrate (110), mechanical cardiac activity sensor (120), electrical cardiac activity sensor (130), and controller (140). In some variations, the substrate (110) may comprise a flexible biocompatible material. For example, the substrate (110) may comprise a silicone or any suitable plastic such as low durometer urethanes, nylons, and polyethylenes.

The substrate (110) may define a first enclosure (112), a second enclosure (114), and a third enclosure (116). The enclosures (112, 114, 116) may be configured to hold the electronic components of the cardiac monitor. For example, the enclosures (112, 114, 116) may be configured to hold one or more components including a mechanical cardiac activity sensor (120) (e.g., accelerometer), an electrical cardiac activity sensor (130, 132) (e.g., electrode), a controller (140) (comprising, for example, a processor and a memory), a communication device (e.g., transceiver), and a power source (e.g., battery). As depicted in FIG. 1D, in some variations, the mechanical cardiac activity sensor (120) and the controller (140) may be disposed in the first enclosure (112), a first electrical cardiac activity sensor (130) may be disposed in the second enclosure (114), and a second electrical cardiac activity sensor (132) may be disposed in the third enclosure (116).

The substrate (110) may comprise a first side (e.g., flat side) that is configured to contact or otherwise face toward the skin of user when the cardiac monitor (100) is in use, and a second side opposite the first side that is configured to face away from the user in use. Each of the cardiac activity sensors (120, 130, 132) may be disposed on the first side of the substrate (110), which may be configured to contact a skin of the user via a hydrogel of an adhesive portion (not shown in FIG. 1). This configuration may allow the sensors (120, 130, 132) to non-invasively generate cardiac data continuously without impairing user movement and function. The second side of the substrate (110) may be configured to protect the cardiac activity sensors (120, 130, 132) from damage. The second enclosure (114) may be positioned on a first portion (e.g., left side) of the cardiac monitor (110), the third enclosure (116) may be positioned on a second portion (e.g. right side) of the cardiac monitor (110) and the first enclosure (112) may be positioned between the second and third enclosures (114, 116). Thus, the first and second electrical cardiac activity sensors (130, 132) may be spaced apart from one another. In some variations, the sensors (120, 130, 132) may be disposed along a longitudinal axis of the substrate (110). Additionally, while the first enclosure (112) is depicted as having a square or rectangular shape, and the second and third (114, 116) enclosures are depicted as having a circular shape, this need not be the case. In other variations, the enclosures may have different shapes, including, for example geometric shapes (e.g., sphere, polygon), symbols (e.g., letters, numbers, logo), combinations thereof, and the like.

As mentioned above, the cardiac monitor (110) may comprise a mechanical cardiac activity sensor (120) configured to measure and/or generate mechanical cardiac data and an electrical cardiac activity sensor (130, 132) configured to measure and/or generate electrical cardiac data. In some variations, the electrical cardiac data may comprise a time-dependent electrocardiograph waveform that represents the electrical activity of the heart, and the mechanical cardiac data may comprise three time-dependent accelerometer waveforms along orthogonal axes corresponding to the mechanical dynamics of the heart. In some variations, the mechanical cardiac activity sensor (120) may be an accelerometer and the electrical cardiac activity sensor (130, 132) may be an electrode. In some variations, the mechanical cardiac activity sensor (120) may be a seismocardiogram (SCG) sensor, and the electrical cardiac activity sensor (130, 132) may be electrocardiogram (ECG) electrode. In some variations, the electrical cardiac activity sensors (130, 132) may have a measurement range between about −5 mV and about 5 mV with a resolution of about 0.000265 mV. In some variations, the mechanical cardiac activity sensor (120) may comprise a micro-electric-mechanical system (MEMS) device. Additionally or alternatively, the mechanical cardiac activity sensor (120) may be configured to measure acceleration between about −4 g and +4 g with a resolution of about 0.488 mg, measure acceleration between about −2 g and +2 g, measure acceleration between about −8 g and +8 g, measure acceleration between about −16 g and +16 g, including all values and sub-ranges in-between. For example, the mechanical cardiac activity sensor (120) may be a 14-bit sensor.

In some variations, the cardiac monitor may comprise one or more visual indicators (e.g., light-emitting diodes) configured to convey an operational status of the cardiac monitor (e.g., ON, recharging, measuring cardiac data, error, low power).

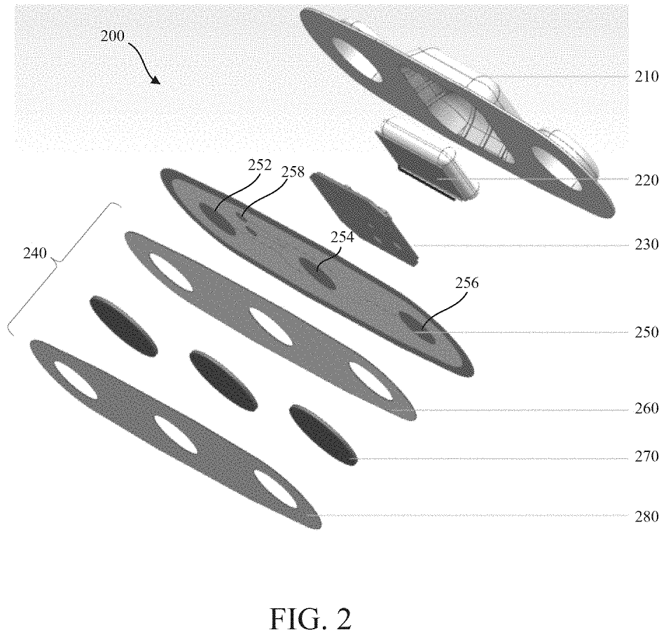

FIG. 2 is an exploded perspective view of an illustrative variation of a cardiac monitor (200). As depicted there, the cardiac monitor (200) may comprise a substrate (210), a battery (220), a controller (230), an adhesive portion (240), a sensor layer (250), electrical cardiac activity sensors (252, 256), a mechanical cardiac activity sensor (254), a connector (258), a first adhesive layer (260), hydrogels (270), and a second adhesive layer (280). The cardiac monitor (200) may comprise a substrate (210), a battery (220), and a controller (230). The battery (220) and the controller (230) may be disposed in an enclosure of the substrate (210). The controller (230) may comprise, for example, a processor, a memory, and an analog-to-digital converter (ADC) circuit. The cardiac monitor may further comprise a communication device (not shown in FIG. 2) comprising an antenna. In some variations, the controller (230) may be configured to control one or more of logic, signal, and data processing, analysis, estimation, communication, calibration, diagnostics, storage, encryption, authentication, and the like.

The substrate (210) may house the electronic components of the cardiac monitor (200) and may form a durable component of the cardiac monitor (200). In some variations, the substrate (210) may be releasably coupled to an adhesive portion (240), which may be configured to attach to a skin of a user for a predetermined time period (e.g., up to about 24 hours, up to about 48 hours, up to about 72 hours, up to about 96 hours, including all values and sub-ranges in-between). The substrate (210) may be a reusable portion of the cardiac monitor (200) and the adhesive portion (240) may be disposable and replaced as desired. In some variations, the substrate (210) may be provided separately from the adhesive portion (240).

As mentioned previously, the cardiac monitor (200) may comprise a sensor layer (250), which may comprise one or more sensors, and may be coupled to, or integrally formed with, the substrate (210). The sensor layer (250) may be flexible and may composed of a thermoplastic polymer including, for example, polyethylene terepthalate (PET), nylon, urethane, or polyethylene (PE). In some variations, the sensor layer (250) may be adhered to the substrate (210) to form a water-impermeable seal. A set of electrodes (252, 256) and an accelerometer (254) may be disposed spaced apart in the sensor layer (250). The electrodes (252, 256) may be composed of electrically conductive pads configured to contact the skin of the user through the adhesive portion (240). For example, in some variations, the electrodes (252, 256) may comprise copper pad having a thickness of between about 0.5 mm and about 3 mm The sensors (252, 254, 256) may be coupled to the controller (230) via electrical leads (not shown) in the sensor layer (250). In some variations, the sensor layer (250) may comprise a connector (258) such as a set of electrical pins (e.g., charging pins) configured to connect to a power source (e.g., dock). For example, the connector (258) may be configured to electrically couple to the battery (220) for recharging and to the controller (230) for communication (e.g., data transfer).

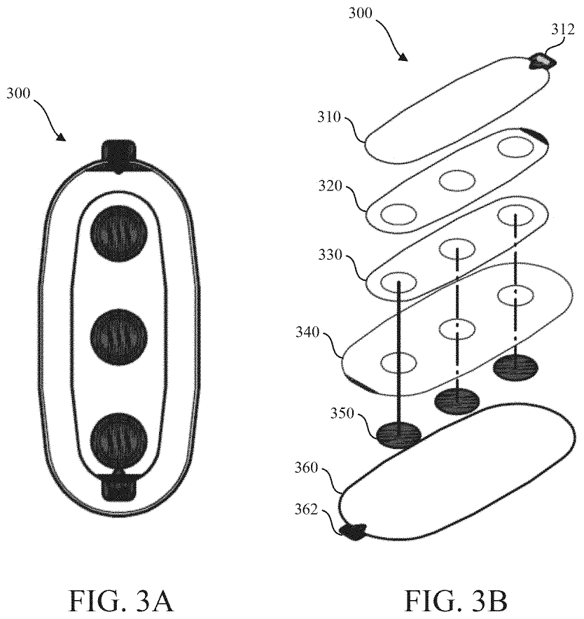

The adhesive portion (240) may include a plurality of adhesive layers. For example, in some variations, the adhesive portion (240) may comprise a first adhesive layer (260) and a second adhesive layer (280). The first adhesive layer (260) may be configured to releasably attach to the sensor layer (254) and the second adhesive layer (280) may be configured to releasably attach to a skin of a user. One or more hydrogels (270) may be coupled between the first adhesive layer (260) and a second adhesive layer (280), and the hydrogel may be configured to serve as a signal interface between the sensor layer (254) and skin. Each of the first and second adhesive layers (260, 280) may define openings aligned with the sensors disposed in the sensor layer (250). The hydrogels (270) may be configured to contact and align with the sensors in the sensor layer (250) and fit within the openings of the first and second adhesive layers (260, 280). When the cardiac monitor (200) is attached to a skin of the user, the sensors (252, 254, 256) may contact the skin through the hydrogel (270). As will be described in more detail with respect to FIGS. 3A and 3B, the adhesive portion may comprise one or more release liners, which may aid in storage and transport of the adhesive portion by covering the adhesive before use. For example, one or more release liners may be removed prior to application to the cardiac monitor and skin of a user.

Turning to FIGS. 3A and 3B, shown there are a plan view of a variation of an adhesive portion (300), and an exploded perspective view of the adhesive portion (300), respectively. The adhesive portion (300) may comprise a first release layer (310) (e.g., a release liner), a first adhesive layer (320), a second adhesive layer (330), a third adhesive layer (340), a set of hydrogels (350), and a second release layer (360). In some variations, the first adhesive layer (320) may be configured to releasably attach to a substrate of the cardiac monitor (not shown) and the third adhesive layer (340) may be configured to releasably attach to a skin of a user (not shown). Each of the first, second, and third adhesive layers (320, 330, 340) may define openings that may align with the sensors of a corresponding cardiac monitor. The hydrogels (350) may be configured to align with openings in the adhesive layers (320, 330, 340). The hydrogels (350) may be configured to releasably attach to a skin of a user and the sensors of the cardiac monitor, may facilitate signal measurement between a sensor layer and skin of the user. For example, when the cardiac monitor is attached to the skin of the user, the sensors of a sensor layer may receive signals from the skin through the hydrogel (350) disposed between the sensors and the skin. The adhesive layers (320, 330, 340) may comprise any biocompatible adhesive. Adhesive layers may comprise thermoplastic thin-film carriers such as polyester (PET) with silicone or acrylic adhesive.

A user may apply the adhesive portion (300) to the cardiac monitor and the skin prior to measuring cardiac data. For example, to releasably couple the cardiac monitor to the skin, the first release layer (310) may be peeled away from the first adhesive layer (320) using a first tab (312) and the first adhesive layer (320) may be applied over the sensor layer of the cardiac monitor. The second release layer (360) may then be peeled away from the third adhesive layer (340) using a second tab (362) and the third adhesive layer (340) may be applied onto the skin of the user. However, the release layers (310, 320) may be peeled away and the adhesive layers (320, 340) may be applied in any order.

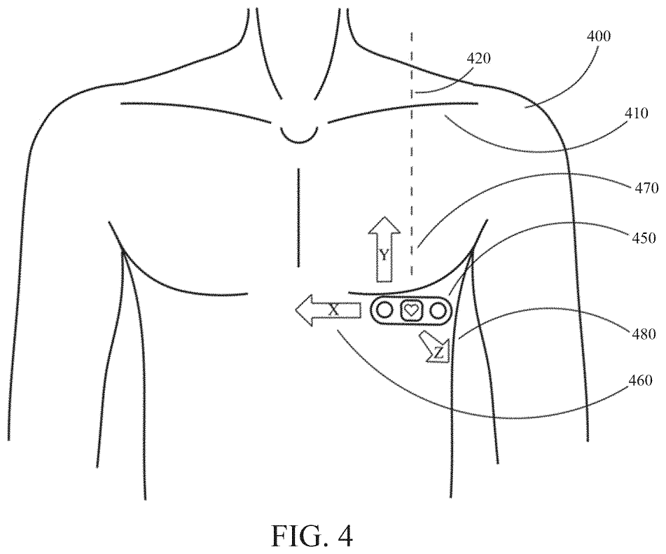

FIG. 4 is a schematic depiction of a cardiac monitor (450) attached to a user (400). As shown in FIG. 4, the cardiac monitor (450) may releasably couple to the skin of the user (400) over the left ribcage, just below the pectoral muscle. The location and orientation of the cardiac monitor (450) attached to the user (400) may determine a strength and/or quality of the signals measured by the cardiac monitor (450). For example, the cardiac monitor (450) may be positioned flat on a lower left portion of the chest, such as atop the apical impulse of the heart, about where the mid-clavicular line (420) of the clavicle bone (410) intersects the fifth intercostal space of the ribcage (not shown) (e.g., at a point of maximum impulse (PMI)). The cardiac monitor (450) may be oriented perpendicular to the mid-clavicular line (420). This placement allows the mechanical cardiac activity sensor to measure high signal-to-noise seismocardiogram waveforms of vibrations as the beating heart hits a chest wall. Furthermore, positioning the cardiac monitor (450) horizontally with respect to the apical impulse allows the electrical cardiac activity sensor to measure high signal-to-noise electrocardiogram waveforms.

In other variations, the cardiac monitor (450) may be releasably coupled to different portions of the chest of a user. For example, in some instances, the cardiac monitor (450) may be coupled to the skin of a user proximate to the sternum or the clavicle (e.g., below the clavicle, left of the sternum) for measurement of mechanical vibrations and, in some variations, at oblique orientations. For example, the cardiac monitor (450) may be positioned on the upper chest for alignment with electrical signal propagation of the heart. The cardiac monitor (450) may be oriented obliquely with respect to the sternal axis between about ±30 degrees, ±45 degrees, and ±60 degrees, including all values and sub-ranges in-between. The cardiac monitor (450) may be coupled to the skin of the user at any location where suitable electrical cardiac data (e.g., ECG waveform) may be measured.

The cardiac monitor (450) attached to the user (400) may synchronously measure semi-continuously or continuously electrocardiograph and seismocardiograph waveforms. In some variations, seismocardiograph waveforms may be measured using a mechanical cardiac activity sensor (e.g., accelerometer). The mechanical cardiac activity sensor may be configured to measure seismocardiograph waveforms in three dimensions. For example, the cardiac monitor (450) may generate a seismocardiogram waveform corresponding to each of the X-axis (460), Y-axis (470), and Z-axis (480). The X-axis (460) corresponds to horizontal motion of the heart, the Y-axis (470) corresponds to vertical motion of the heart, and the Z-axis (480) corresponds to motion along a vector normal to the surface of the ribcage.

### Computing Device

Generally, the computing devices described here may comprise a controller comprising a processor (e.g., CPU) and memory (which can include one or more non-transitory computer-readable storage mediums). The processor may incorporate data received from memory and over a communication channel to control one or more components of the system (e.g., cardiac monitor (510)). The memory may further store instructions to cause the processor to execute modules, processes and/or functions associated with the methods described herein. As used herein, a computing device may refer to any of the computing devices (520, 522), databases (540), and servers (550) as depicted in FIG. 5. In some variations, the memory and processor may be implemented on a single chip. In other variations, they can be implemented on separate chips.

A controller may be configured to receive and process cardiac data from the cardiac monitor and other data (e.g., cohort data, reference blood pressure) from other sources (e.g., computing device (520, 522), database (540), user input). The computing devices may be configured to receive, process, compile, store, and access data. In some variations, the computing device may be configured to access and/or receive data from different sources. The computing device may be configured to receive data directly input and/or measured from a patient. Additionally or alternatively, the computing device may be configured to receive data from separate devices (e.g., a smartphone, tablet, computer) and/or from a storage medium (e.g., flash drive, memory card). The computing device may receive the data through a network connection, as discussed in more detail herein, or through a physical connection with the device or storage medium (e.g. through Universal Serial Bus (USB) or any other type of port). The computing device may include any of a variety of devices, such as a cellular telephone (e.g., smartphone), tablet computer, laptop computer, desktop computer, portable media player, wearable digital device (e.g., digital glasses, wristband, wristwatch, brooch, armbands, virtual reality/augmented reality headset), television, set top box (e.g., cable box, video player, video streaming device), gaming system, or the like.

The computing device may be configured to receive various types of data. For example, the computing device may be configured to receive a patient's personal data (e.g., gender, weight, birthday, age, height, diagnosis date, anniversary date using the device, etc.), a patient's cardiac data (e.g., blood pressure data, heart rate data), general health information of other similarly situated patients (e.g., cohort cardiac data), or any other relevant information. In some variations, the computing device may be configured to create, receive, and/or store patient profiles. A patient profile may contain any of the patient specific information previously described. While the above mentioned information may be received by the computing device, in some variations, the computing device may be configured to process any of the above data from information it has received using software stored on the device itself, or externally.

The processor may be any suitable processing device configured to run and/or execute a set of instructions or code and may include one or more data processors, image processors, graphics processing units, physics processing units, digital signal processors, and/or central processing units. The processor may be, for example, a general purpose processor, Field Programmable Gate Array (FPGA), an Application Specific Integrated Circuit (ASIC), and/or the like. The processor may be configured to run and/or execute application processes and/or other modules, processes and/or functions associated with the system and/or a network associated therewith. The underlying device technologies may be provided in a variety of component types (e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and/or the like.

In some variations, the memory may include a database (not shown) and may be, for example, a random access memory (RAM), a memory buffer, a hard drive, an erasable programmable read-only memory (EPROM), an electrically erasable read-only memory (EEPROM), a read-only memory (ROM), Flash memory, and the like. The memory may store instructions to cause the processor to execute modules, processes, and/or functions associated with the communication device, such as cardiac data processing, cardiac parameter estimation, cardiac monitor control, and/or communication. Some variations described herein relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for the specific purpose or purposes.

Examples of non-transitory computer-readable media include, but are not limited to, magnetic storage media such as hard disks, floppy disks, and magnetic tape; optical storage media such as Compact Disc/Digital Video Discs (CD/DVDs); Compact Disc-Read Only Memories (CD-ROMs), and holographic devices; magneto-optical storage media such as optical disks; solid state storage devices such as a solid state drive (SSD) and a solid state hybrid drive (SSHD); carrier wave signal processing modules; and hardware devices that are specially configured to store and execute program code, such as Application-Specific Integrated Circuits (ASICs), Programmable Logic Devices (PLDs), Read-Only Memory (ROM), and Random-Access Memory (RAM) devices. Other variations described herein relate to a computer program product, which may include, for example, the instructions and/or computer code disclosed herein.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

In some variations, the computing device (520, 522) may further comprise a communication device configured to permit a user and/or health care professional to control one or more of the devices of the system. The communication device may comprise a network interface configured to connect the computing device to another system (e.g., Internet, remote server, database) by wired or wireless connection. In some variations, the computing device (520, 522) may be in communication with other devices via one or more wired and/or wireless networks. In some variations, the network interface may comprise a radiofrequency receiver, transmitter, and/or optical (e.g., infrared) receiver and transmitter configured to communicate with one or more devices and/or networks. The network interface may communicate by wires and/or wirelessly with one or more of the cardiac monitor (510), network (530), database (540), and server (550).

The network interface may comprise RF circuitry configured to receive and send RF signals. The RF circuitry may convert electrical signals to/from electromagnetic signals and communicate with communications networks and other communications devices via the electromagnetic signals. The RF circuitry may comprise well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth.

Wireless communication through any of the computing and measurement devices may use any of plurality of communication standards, protocols and technologies, including but not limited to, Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (WiFi) (e.g., IEEE 802.11a, IEEE 802.11b, IEEE 802.11g, IEEE 802.11n, and the like), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol. In some variations, the devices herein may directly communicate with each other without transmitting data through a network (e.g., through NFC, Bluetooth, WiFi, RFID, and the like).

The communication device may further comprise a user interface configured to permit a user (e.g., subject, predetermined contact such as a partner, family member, health care professional, etc.) to control the computing device. The communication device may permit a user to interact with and/or control a computing device directly and/or remotely. For example, a user interface of the computing device may include an input device for a user to input commands and an output device for a user to receive output (e.g., blood pressure readings on a display device).

An output device of the user interface may output blood pressure estimates and may comprise one or more of a display device and audio device. Data analysis generated by a server (550) may be displayed by the output device (e.g., display) of the computing device (520, 522). Data used in blood pressure estimation such as reference blood pressure of a user, cohort cardiac data, and cardiac data from the cardiac monitor (510) may be received through the network interface and output visually and/or audibly through one or more output devices of the computing device (520). In some variations, an output device may comprise a display device including at least one of a light emitting diode (LED), liquid crystal display (LCD), electroluminescent display (ELD), plasma display panel (PDP), thin film transistor (TFT), organic light emitting diodes (OLED), electronic paper/e-ink display, laser display, and/or holographic display.

An audio device may audibly output cardiac data, cardiac parameter data, system data, alarms and/or notifications. For example, the audio device may output an audible alarm when an estimated blood pressure falls outside a predetermined range or when a malfunction in the cardiac monitor (510) is detected. In some variations, an audio device may comprise at least one of a speaker, piezoelectric audio device, magnetostrictive speaker, and/or digital speaker. In some variations, a user may communicate with other users using the audio device and a communication channel For example, a user may form an audio communication channel (e.g., VoIP call) with a remote health care professional.

In some variations, the user interface may comprise an input device (e.g., touch screen) and output device (e.g., display device) and be configured to receive input data from one or more of the cardiac monitor (510), network (530), database (540), and server (550). For example, user control of an input device (e.g., keyboard, buttons, touch screen) may be received by the user interface and may then be processed by processor and memory for the user interface to output a control signal to the cardiac monitor (510). Some variations of an input device may comprise at least one switch configured to generate a control signal. For example, an input device may comprise a touch surface for a user to provide input (e.g., finger contact to the touch surface) corresponding to a control signal. An input device comprising a touch surface may be configured to detect contact and movement on the touch surface using any of a plurality of touch sensitivity technologies including capacitive, resistive, infrared, optical imaging, dispersive signal, acoustic pulse recognition, and surface acoustic wave technologies. In variations of an input device comprising at least one switch, a switch may comprise, for example, at least one of a button (e.g., hard key, soft key), touch surface, keyboard, analog stick (e.g., joystick), directional pad, mouse, trackball, jog dial, step switch, rocker switch, pointer device (e.g., stylus), motion sensor, image sensor, and microphone. A motion sensor may receive user movement data from an optical sensor and classify a user gesture as a control signal. A microphone may receive audio data and recognize a user voice as a control signal.

A haptic device may be incorporated into one or more of the input and output devices to provide additional sensory output (e.g., force feedback) to the user. For example, a haptic device may generate a tactile response (e.g., vibration) to confirm user input to an input device (e.g., touch surface). As another example, haptic feedback may notify that user input is overridden by the computing device.

### Network

In some variations, the systems and methods described herein may be in communication with other computing devices via, for example, one or more networks, each of which may be any type of network (e.g., wired network, wireless network). The communication may or may not be encrypted. A wireless network may refer to any type of digital network that is not connected by cables of any kind. Examples of wireless communication in a wireless network include, but are not limited to cellular, radio, satellite, and microwave communication. However, a wireless network may connect to a wired network in order to interface with the Internet, other carrier voice and data networks, business networks, and personal networks. A wired network is typically carried over copper twisted pair, coaxial cable and/or fiber optic cables. There are many different types of wired networks including wide area networks (WAN), metropolitan area networks (MAN), local area networks (LAN), Internet area networks (IAN), campus area networks (CAN), global area networks (GAN), like the Internet, and virtual private networks (VPN). Hereinafter, network refers to any combination of wireless, wired, public and private data networks that are typically interconnected through the Internet, to provide a unified networking and information access system.

Cellular communication may encompass technologies such as GSM, PCS, CDMA or GPRS, W-CDMA, EDGE or CDMA2000, LTE, WiMAX, and 5G networking standards. Some wireless network deployments combine networks from multiple cellular networks or use a mix of cellular, Wi-Fi, and satellite communication.

The communication between the processor and the cardiac monitor may or may not be performed in real-time as the cardiac data is received or recorded. The processor may be located in the same housing as the cardiac monitor, or in a separate housing in the same room or building as the cardiac monitor. The processor may also be located in a remote location from the cardiac monitor (e.g., a different building, city, country).

### Dock

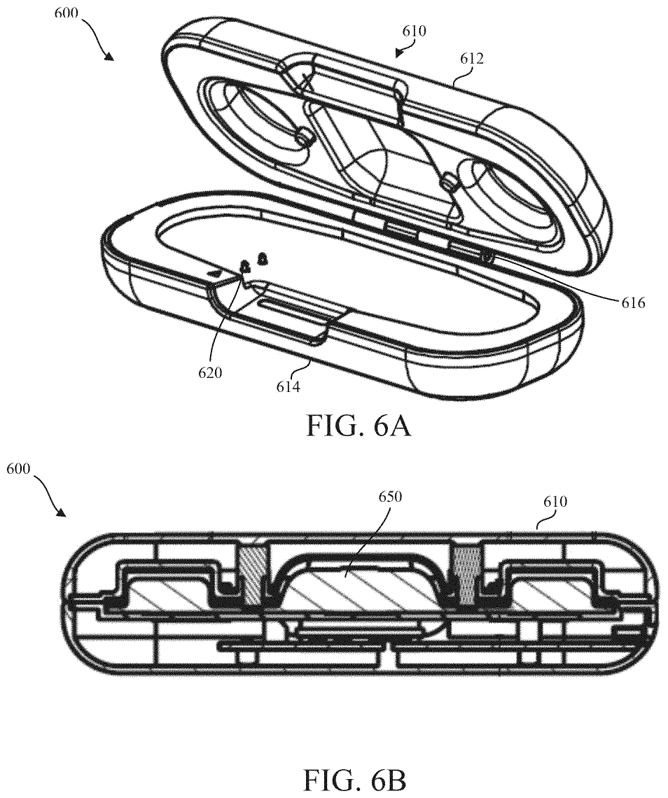

In some variations, the cardiac monitoring system (500) may include a dock (512), which may be configured to hold and secure the cardiac monitor (510) for one or more of charging, storage, transportation, and handling when not in use. FIGS. 6A and 6B are respective perspective and cross-sectional side views of a variation of a dock (600). The dock (600) may comprise an enclosure (610) comprising an inner recess shaped to hold a cardiac monitor (650). The enclosure (610) may comprise a first portion (610) and a second portion (620) rotatably coupled by a hinge (616). The dock (600) may be configured to hold, for example, the reusable portion of the cardiac monitor (650). For example, the first portion (610) may comprise a plurality of recesses, each corresponding to the size and shape of an enclosure in the substrate of the cardiac monitor (650). For example, the first portion (610) may comprise a first circular recess, a second circular recess, and a square recess between the first and second circular recesses. The second portion (620) may comprise a recess corresponding to the size and shape of the first side (e.g., user-facing) of the cardiac monitor (650). For example, the second portion (620) may comprise an elliptical recess. In some variations, the dock (600) may comprise a second enclosure (not shown) configured to hold one or more adhesive portions separate from the cardiac monitor (650). For example, the second enclosure may define an opening or compartment in the first portion (612) or the second portion (620) of the dock (600).

In some variations, the dock (600) may comprise a power and/or data connector (620) configured to establish a power and/or data connection with the cardiac monitor (650). The dock (600) may be configured to recharge an internal power source of the cardiac monitor (600) by electrically coupling a connector (620) of the dock (600) to a corresponding connector of the cardiac monitor (650). For example, the power connector (620) may comprise one or more of a set of pogo pins, a USB connector, or any suitable connector. The dock (600) may comprise one or more of an internal power source (e.g., lithium-ion battery) and/or connecter configured to couple to a power source. In some variations, the dock (600) may comprise one or more visual indicators (e.g., light-emitting diodes) configured to convey an operational status of the dock (600) (e.g., ON, recharging cardiac monitor, error, low power). For example, an LED of the dock (600) may be configured to emit red light when a cardiac monitor (650) placed in the dock (600) is charging and emit green light when a charging process has completed.

In some variations, the dock (600) may comprise a communication device configured to form a communication channel with one or more of the cardiac monitor (650) and a computing device. For example, the dock (600) may be configured to form a wired or wireless communication channel to receive cardiac data from the cardiac monitor (650). The dock (600) may be configured to transmit and/or receive data with the cardiac monitor (650) including, but not limited to, user data, cardiac data, configuration data, device data, firmware updates, and the like using the communication channel

### II. Methods

Also described here are methods for non-invasively monitoring a physiological characteristic of a patient using the systems and devices described herein. In particular, the systems, devices, and methods described herein may be used to accurately estimate and track values of a physiological characteristic, such as, for example, blood pressure. Conventional methods of determining blood pressure often require use of tools, for example, a blood pressure cuff and a stethoscope, and that can be cumbersome, difficult to for a non-medical professional to use, and difficult to use outside of a clinical setting. These conventional methods also fail to provide continuous or semi-continuous monitoring, require an interruption in normal daily activities, and lack privacy. The methods described herein are advantageous relative to conventional methods in several ways. For example, use of the cardiac monitors described herein provides non-medical professionals the ability to accurately, continuously or semi-continuously, non-invasively, and discretely determine and track blood pressure without interrupting their daily activities. The devices, systems, and methods, are easy for a non-medical profession to use and require little training, allow for a private blood pressure determination while in public, and provide a comfortable and portable way to determine and track an important health indicator. Moreover, because the cardiac monitors may be flexible and may comprise or otherwise be used with biocompatible adhesive, the cardiac monitors may remain comfortably, continuously wearable for several days to several weeks without interfering in a user's activities and without significant up-keep.

Additionally, the methods described herein advantageously provide an accurate determination of a user's blood pressure regardless of a user's age, gender, or current activity level. As will be described in more detail herein, the methods described here may utilize a change in one or more mechanical cardiac parameters to estimate a user's blood pressure, and, in contrast to conventional techniques, are not reliant on the calculation of a time difference between cardiac events obtained from multiple signals or signals indicating such (such as, methods utilizing pulse transit time). Accordingly, the methods described herein may accurately, and in some instances, more accurately estimate blood pressure for users of different genders (male or female) and ages (under 20, 20-29, 30-39, 40-49, 50-59, 60-69, 70+), as well as when users have engaged in different activities (e.g., during rest, during light exercise, during strenuous exercise, during a mentally strenuous tasks). Furthermore, the methods described are differentiated from methods utilizing a combination of proximal and distal pulse measurements for PTT, wherein the location of the distal pulse measurement relative to the heart level may result in poor blood pressure estimation.

The determined physiological characteristic, such as blood pressure, may be used in a variety of ways. For example, as will be described herein, the physiological characteristic may be displayed to a user on a computing device and/or may be stored on the computing device or on a server for later viewing on the computing device. In some instances, the determined physiological characteristic may be used in conjunction with data from other devices or mobile applications, e.g., an activity or fitness tracker, a sleep tracker, a glucometer, an internet-enabled scale and/or body composition device, a meditation tracker, or the like, to provide a user or a health care professional with a more comprehensive view of the user's health. In some variations, the determined physiological characteristic, cardiac data, mechanical cardiac parameters and/or electrical cardiac parameters may be exported to or used by mobile applications or devices that may analyze them in conjunction with other health related data (e.g., activity data, fitness data, sleep data, weight, body fat percentage, temperature, blood glucose, or the like). In some variations, the values of the determined physiological characteristic, and/or trends established from the values, may be used to predict a future health (e.g., cardiac) event. Additionally or alternatively, in some variations, the physiological characteristics may be utilized to remotely monitor and/or manage a user. For example, users with known risk factors for cardiovascular disease or other heart disease may be more actively and comprehensively monitored using mobile applications. Health care providers, for example, primary care physicians and/or cardiac care teams, may use the information to adjust medication regimen, clinical assessments, and/or to inform therapy decision-making. In another example, users with risk factors for hypertension or concomitant diseases may be monitored to inform clinicians of therapy efficacy and/or to prevent further progression of disease.

### Estimating Blood Pressure

When the physiological characteristic is blood pressure, the methods for estimating a user's blood pressure may generally comprise receiving mechanical cardiac data of a user measured using an accelerometer and generating a mechanical cardiac parameter value for a first time period and a second time period using the mechanical cardiac data. The blood pressure of the user may be estimated based on a change in the mechanical cardiac parameter value between the first and second time periods. In some variations, the blood pressure estimate may be further based on a regression model that receives as input a reference blood pressure of a user and cohort cardiac data associated with the user. It should be appreciated that any of systems and devices described herein may be used in the methods described here.

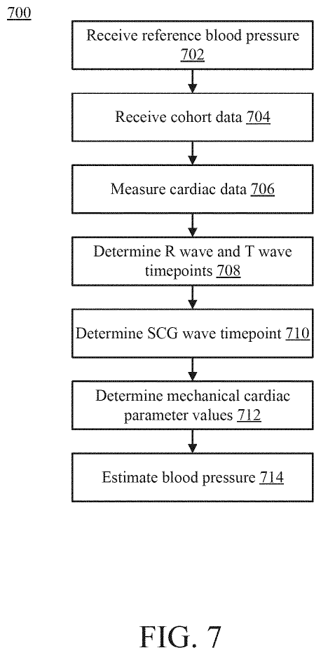

FIG. 7 is a flowchart depicting an illustrative variation of a method of estimating blood pressure (700). In the variation depicted in FIG. 7, the method may comprise receiving a reference blood pressure (702), receiving cohort cardiac data (704), measuring cardiac data (706), determining R wave and T wave timepoints (708), determining SCG wave timepoints (710), determining mechanical cardiac parameter values (712), and estimating a blood pressure of a user (714). As used herein, blood pressure may include one or more of systolic blood pressure and diastolic blood pressure. Moreover, reference blood pressure refers to a blood pressure measurement measured using a conventional blood pressure monitoring device such as a sphygmomanometer and blood pressure cuff as described herein. The reference blood pressure may be measured once, or at predetermined intervals (e.g., weekly, biweekly, monthly), using, for example, a blood pressure cuff. The reference blood pressure measurement may be stored in a computing device such as any of the computing devices described herein. For example, a health care provider of a user may input the reference blood pressure into a health care provider computing device. The device used to estimate the blood pressure (e.g., computing device, cardiac monitor) may receive the reference blood pressure data from any computing device storing the reference blood pressure of the user. In some variations, the reference blood pressure data may include measurements taken under different physiological conditions such as a resting condition and non-resting conditions such as during various levels of physical and mental activity. In some variations, a single blood pressure measurement taken at rest may be utilized as the reference blood pressure, while in other variations, multiple blood pressure measurements (e.g., two, three, four, five, six, or more) may be utilized to determine the reference blood pressure. In variations in which multiple blood pressure measurements are used, the measurements may all be taken under the same condition (e.g., at rest), or the measurements may be taken under a combination of different conditions (e.g., at rest, during or after physical activity, during or after mental activity). In variations in which a reusable cardiac monitor is used, a new blood pressure measurement may be taken upon each new application of the cardiac monitor to a user's skin (e.g., application of new adhesive) and this new measurement may be utilized alone or in combination with prior reference blood pressure measurements.

In some variations, a reference blood pressure of the user may be measured using an oscillometric blood pressure monitor including a blood pressure cuff (e.g., sphygmomanometer). For example, the cuff may be coupled to a left arm of the user at the same height as the heart. The user may be seated with feet planted on the floor and with the left hand resting palm up on a surface such as a table. In some variations, the reference blood pressure may be measured for about one minute while the user is resting comfortably. In other variations, a reference blood pressure may be measured under non-resting conditions such as during various levels of physical and mental activity. For example, blood pressure may be measured under a plurality of conditions including one or more of when the user is solving arithmetic problems, exposed to low intensity stimulus (e.g., listening to relaxing music, viewing a beach scene using a virtual reality headset), exposed to high intensity stimulus (e.g., experiencing a roller coaster ride using a virtual reality headset), and the like. This may allow the blood pressure estimation to be calibrated to an activity level of a user when a cardiac monitor is attached to the user.

In step 704, the device used to estimate the blood pressure (e.g., computing device, cardiac monitor) may receive and use, or store for later use, the cohort cardiac data associated with the user. In some variations, the user may be classified into a cohort (e.g., demographic group, peer group), which may include a set of users grouped by one or more of age, gender, race, and body mass index. For example, the user may input demographic data into a computing device including information such as their age, gender, race, weight, height, body mass index, and the like to determine the cohort of a user. In some variations, the cohort cardiac data may comprise a look up (LUT) table stored in memory and/or an algorithm stored in memory. The cohort cardiac data may comprise values for one or more of the electrical and mechanical cardiac parameters described herein such as heart rate, blood pressure, SCG1 and SCG2 wave timepoints, SCG wave amplitude, and the like. In some variations, the cohort cardiac data may be pre-programmed and stored in memory or may be received (e.g., updated) over a communication channel.

In step 706, a cardiac monitor may measure the cardiac data of a user. As described with respect to FIG. 4, a cardiac monitor may be releasably attached flat to the skin of the user over the left ribcage just below the pectoral muscle (e.g., at the intersection of the mid-clavicular line of the clavicle bone to the fifth intercostal space of the ribcage). In some variations, it may be beneficial to attach the cardiac monitor to an area of skin that is clean and substantially free of hair. The sensors of the cardiac monitor may be configured to measure electrical and mechanical cardiac data, such as ECG signal waveforms and SCG signal waveforms, continuously or at pre-determined intervals while the user performs any of their daily activities.

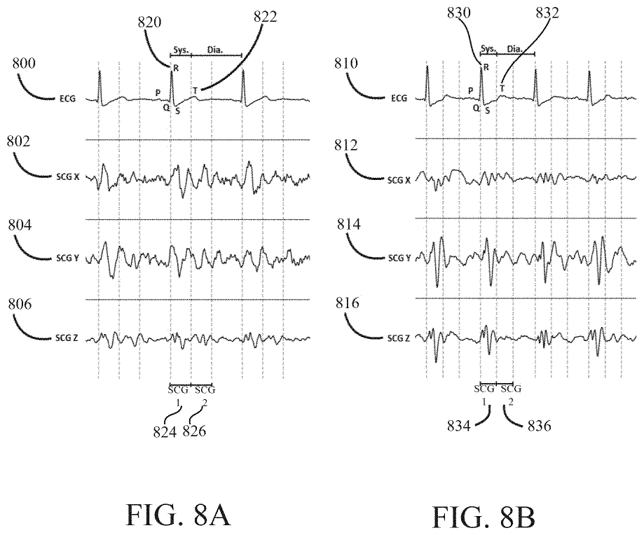

As mentioned above, the measured cardiac data may comprise electrical and mechanical signal waveforms. FIGS. 8A and 8B are illustrative variations of ECG waveforms (800, 810) of electrical cardiac activity and SCG waveforms (802, 804, 806, 812, 814, 816) of mechanical cardiac activity measured over a respective first time period (FIG. 8A) and a second time period (FIG. 8B). Measurement of cardiac activity over first and second time periods allows for comparative hemodynamic analysis useful in the blood pressure estimation processes described herein. FIG. 8A shows ECG and SCG waveforms measured synchronously over a first time period and includes an ECG waveform (800), X-axis SCG waveform (802), Y-axis SCG waveform (804), and Z-axis SCG waveform (806). Likewise, FIG. 8B shows ECG and SCG waveforms measured synchronously over a second time period and includes an ECG waveform (810), X-axis SCG waveform (812), Y-axis SCG waveform (814), and Z-axis SCG waveform (816). The measurement of SCG waveforms along the X, Y, and Z axis allows a three-dimensional SCG waveform to be generated that is invariant to position. This may improve cardiac parameter estimation and/or reduce the need to place the cardiac monitor at an optical location in the chest.

ECG waveforms (800, 810) correspond to electrical activity of the heart. For example, R waves (820, 830) corresponds to the depolarization of the ventricles and contraction of the large ventricular muscles. The T waves (822, 832) correspond to the repolarization of the ventricles. The R waves (820, 830) and T waves (822, 832) divide the ECG waveforms (800, 810) into systole and diastole time periods. The systole and diastole segments of the SCG waveforms (802, 804, 806, 812, 814, 816) are associated with turbulent blood flow caused by the heart valves closing. For example, the SCG1 wave (824, 834) corresponds to the closure of the mitral and tricuspid valves during systole. The SCG2 wave (826, 836) corresponds to the closure of the aortic and pulmonary valves during the first half of diastole. Gallop rhythms SCG3 and SCG4 (not shown) may appear during the second half of diastole for users experiencing heart failure or certain other heart conditions.

The ECG and SCG signal waveforms may be generated by the cardiac monitor in any of the known digital ECG and SCG formats, or alternative image formats, such as .jpg, .gif, and the like. In some variations, ECG signal formats may include, but are not limited to, the Standard Communications Protocol for computer assisted ElectroCardiography (SCP-ECG), HL7 annotated ECG (HL7 aECG), Digital Imaging and Communication in Medicine (DICOM) Waveform Supplement 30, and Medical waveform Format Encording Rules (MFER). Any known digital ECG and SCG format may be utilized in conjunction with the devices and methods described herein. In some variations, the ECG signal data may be recorded at a rate in the range of about 125 Hz to about 250 Hz, about 500 Hz to about 1 kHz, and about 1 kHz to about 16 kHz with a resolution in the range of about 8 bit to about 64 bit, about 16 bit to about 32 bit, and about 24 bit.

The ECG and SCG signal waveforms measured in step 706 may be affected by noise from one or more different sources, including physiological and non-physiological sources. Examples of physiological noise include axis shift, biphasic QRS morphology, and QRS amplitude variations. Non-physiological noise sources may include 50/60 Hz electric power lines, electrode motion artifacts, myogram, and baseline wander. In some variations, the measured ECG and SCG signal waveforms may be processed to reduce noise. For example, the cardiac monitor may pre-process the measured cardiac data before transmitting the data to a computing device for blood pressure estimation. In some variations, the ECG waveform may be pre-processed to filter out noise and increase a signal-to-noise ratio. For example, the ECG waveform may be processed by an infinite impulse response (IIR) bandpass filter configured to suppress frequencies other than between about 2 Hz and about 40 Hz such that low-frequency noise (due to baseline wander and respiration) and high-frequency noise (due to motion artifacts) are removed from the ECG waveform.

In some variations, one or more of the raw cardiac data and pre-processed cardiac data may be transmitted from the cardiac monitor to the computing device using a communication channel. The communication channel may be established between the cardiac monitor and the computing device. The communication channel may be a wired or wireless connection and may use any communication protocol including but not limited to those described herein such as Bluetooth and NFC. The communication channel may be established at predetermined intervals based on one or more of time (e.g., hourly, daily, weekly, etc.), device usage (e.g., when measuring cardiac data, upon device power on, before entering sleep mode, memory usage, battery level, establishment of a communication channel, etc.), request for connection, and the like.

Additionally or alternatively, a communication channel may be manually established by a user at any desired time. The cardiac monitor may establish the communication channel directly or indirectly with one or more computing devices (e.g., smartphone, dock, database, remote server, Internet, and the like) as described herein. For indirect connections, the intermediary device(s) may establish additional communication channels. For example, a dock may establish a connection to a smartphone to initially transfer cardiac data. The smartphone may then transfer the cardiac data to a cloud database and/or any other computing device (e.g., remote server). In some variations, the cardiac monitor may attempt to find a computing device to establish a communication channel for a predetermined amount of time (e.g., one minute) after measuring cardiac data. The cardiac monitor may preferably connect to a recognized and/or authorized computing device such as a patient's smartphone, laptop, and/or desktop computer.

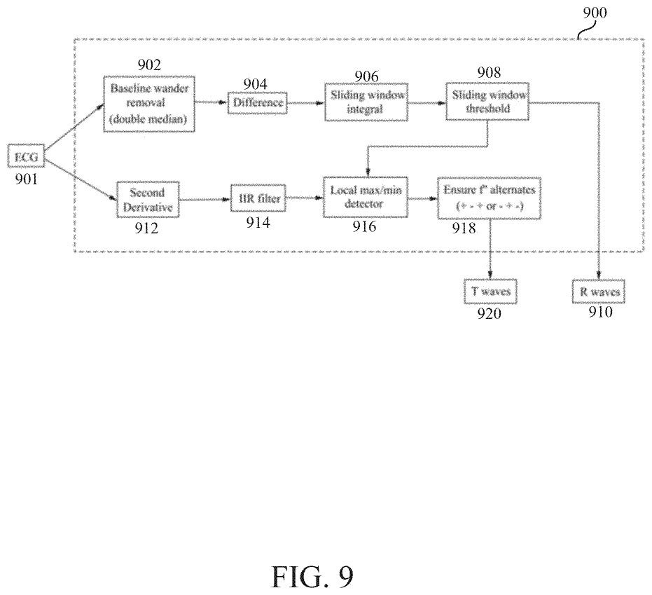

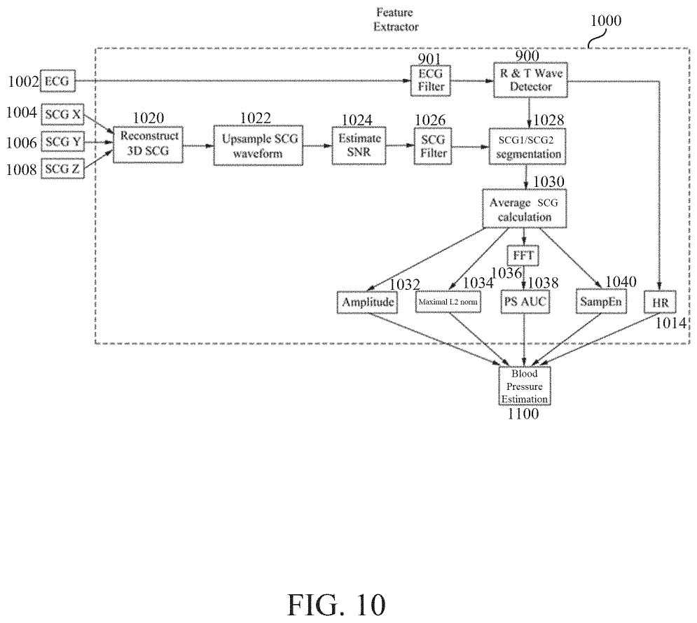

Before mechanical cardiac parameter values are determined using the measured ECG and SCG waveforms, characteristics of the SCG waveform such as the timepoints of the SCG features may be determined. In some variations, the SCG wave features may be accurately determined using the R wave and T wave timepoints. In step 708, a computing device may be used to determine R wave and T wave timepoints in the measured ECG waveforms. FIGS. 9 and 10 are flowcharts directed to the processing of ECG and SCG waveforms used to determine electrical and mechanical cardiac parameter values used for estimation of a physiological characteristic. In some variations, ambient noise and motion artifacts in the measured signal waveforms may be detected and removed by the cardiac monitor.

FIG. 9 is a flowchart describing a variation of a method of determining electrical cardiac parameter values including R wave and T wave timepoints (900). In the method shown there, the ECG waveform may be pre-processed (901) to filter out noise and increase a signal-to-noise ratio. The ECG waveform (e.g., filtered ECG waveform) may then be input in parallel to an R-wave detection process (902, 904, 906, 908, 910) and a T-wave detection process (912, 914, 916, 918, 920). In the R-wave detection process, the ECG waveform may be input to a filter (902), such as a double median filter, that removes baseline wander to suppress non-QRS portions of the ECG waveform. For example, the double median filter may include window sizes of 7 and 11 samples. A successive difference may be calculated (904) between each sample. Sliding window integration (906) may be performed on the successive difference where the result at each iteration is the sum of the values in a predetermined window size. In some variations, R wave timepoints may be determined (908) when: 1) the integral value at a predetermined index value is the maximum within a predetermined window (e.g., 200 ms) centered on the predetermined index value; and 2) the integral value at the predetermined index value is larger than μ+σ, where μ and σ respectively represent the average and standard deviation of the integral value over a predetermined time period (e.g., the last three seconds). The resulting R wave timepoints may be output (910) and may be used as input to the local min/max detector (916).

In the T-wave detection process, a coarse second derivative (e.g., with h=7) of the ECG waveform (901) may be calculated (912). The second derivative may be calculated using a centered difference quotient given by equation (1):

$${f}^{″} ( x ) \approx \frac{f ( x + h ) - 2 f ( x ) + f ( x - h )}{{h}^{2}} \qquad ( 1 )$$

In equation (1), f represents the ECG waveform and f′″ represents its second derivative. An infinite impulse response (IIR) filter (914) may be applied to the output of the second derivative (912), the output of which may be input to a local min/max detector (916). The local min/max detector (916) may also receive the R wave timepoints (910) as input from the sliding window threshold (908). The R wave timepoints (910) may be used to define the search windows in which to detect T waves. Within these search windows, minima and maxima may be detected (916). When the sign of the minima and maxima of the second derivatives f′″ alternate appropriately (i.e. goes from positive to negative to positive or negative to positive to negative) (918), the timepoint with the largest second derivative (in absolute value) may be output as the T wave timepoint (920). The R wave timepoints and T wave timepoints may be subsequently used in determining SCG1 or SCG2 wave timepoints in corresponding SCG waveforms, which may be used to determine mechanical cardiac parameters.

FIG. 10 is a flowchart describing a variation of a method of determining mechanical cardiac parameter values (1000). In the variation shown there, four time-dependent waveforms may be utilized to determine the mechanical cardiac parameter values: ECG waveform (1002); X-axis SCG waveform (1004); Y-axis SCG waveform (1006); and Z-axis SCG waveform (1008). ECG and SCG waveforms may be used in the method of determining mechanical cardiac parameter values (1000) for each time period of interest. Each time period of an ECG and SCG waveform may comprise a plurality of heartbeats (e.g., segments) each having an R wave, T wave, SCG wave, and the like. Since the ECG and SCG waveforms are measured synchronously, the R wave timepoint and T wave timepoint from the ECG waveform correspond to SCG1 wave timepoint and SCG2 wave timepoint as shown in FIGS. 8A and 8B.

The raw ECG waveform (1002) measured using a cardiac monitor may be input to an ECG filter (901) of the cardiac monitor to remove noise and increase the signal-to-noise ratio, as described in detail with respect to FIG. 9. The pre-processed ECG waveform may then be input to R wave and T wave detector (900) and processed as described with respect to FIG. 9. The R wave timepoint and T wave timepoint may be provided as input (1028) as described in more detail herein. The output of the R wave and T wave detector (900) may also be used to calculate a heart rate (HR) (1014). For example, a heart rate (HR) may be calculated in beats per minute using equation (2):

$$HR = 60 \cdot \frac{{f}_{s}}{R R I} \qquad ( 2 )$$

In equation (2), RRI is the average distance between successive R waves, and fₛ is the sampling rate of the ECG sensor in Hertz. In some variations, a mechanical cardiac parameter may include heart rate.

Referring again to FIG. 10, the SCG waveforms may be first processed by generating a position invariant, three-dimensional SCG waveform. For example, the three SCG waveforms (1004, 1006, 1008) may be combined into a joint SCG waveform (1020) (SCG_{xyz}). This may be accomplished using equation (3):

$$S C {G}_{x y z} = \sqrt{S C {G}_{x}^{2} + S C {G}_{y}^{2} + S C {G}_{z}^{2}} . \qquad ( 3 )$$

In some variations, the joint SCG waveform SCG_{xyz} may be up-sampled (1022) to about 500 Hertz using linear interpolation. Up-sampling may be optionally performed to reduce computational load on, for example, the cardiac monitor. The signal-to-noise ratio (SNR) of SCG_{xyz} may be calculated (1024), and the joint seismocardiogram waveform SCG_{xyz} may be filtered using an IIR filter (1026). In some variations, portions of the waveform SCG_{xyz} having an SNR below a pre-determined threshold may be excluded from further processing.

The timepoints of the SCG waves (SCG1 wave, SCG2 wave) of the SCG_{xyz} waveform may then be determined (1028). For example, since the ECG and SCG waveforms are measured synchronously, the R wave timepoints and T wave timepoints from the ECG waveform may be used to aid in detecting and/or identifying the timepoints of the SCG1 and SCG2 waves (1028) within a time period.

In some variations, average SCG1 and SCG2 waves may be calculated across all segments (1030). Utilizing average SCG1 and SCG2 waves may assist in reducing variance due to motion, speech, and respiration. Additionally, in some instances, cubic spline interpolated rescaling to correct for variability in the SCG amplitude due to heavy respiration may be utilized. Averaging the SCG waves may increase the accuracy of the mechanical cardiac parameter value calculation and subsequent blood pressure estimation. The average SCG1 wave may be used when estimating systolic blood pressure, and the average SCG2 wave may be used when estimating diastolic blood pressure.

A plurality of mechanical cardiac parameters may be calculated from the SCG1 and SCG2 waves (e.g., average SCG1 and average SCG2 waves) (1030). For example, the SCG1 and SCG2 waves may be used to calculate the amplitude (1032), the maximal L2 norm (1034), the area under a section of the power spectral density (PS AUC) (1038), the sample entropy (SampEN) (1040), and the like. As used here, the amplitude is the difference between a peak height and a trough height in the average SCG1 wave or SCG2 wave. The maximal L2 norm is the maximum distance between any pair of points on the SCG_{xyz} waveform, and is given by equation (4):

$$Maximal L 2 norm = {max}_{{t}_{1} , {t}_{2}} \sqrt{{\sum }_{i ∈ \{ x , y , z \}} , {( S C {G}_{i} [ {t}_{1} ] - S C {G}_{x} [ {t}_{2} ] )}^{2}} \qquad ( 4 )$$

In equation (4), t₁ and t₂ may range over the length of the average SCG1 wave or SCG2 wave. The area under a section of a power spectral density is given by equation (5):

$$PS AUC = \sum {\vert FFT \vert}^{2} \qquad ( 5 )$$

FFT in equation (5) corresponds to a standard Fast Fourier Transform of the SCG waveform (1036), where the sum is over an adjustable frequency band.

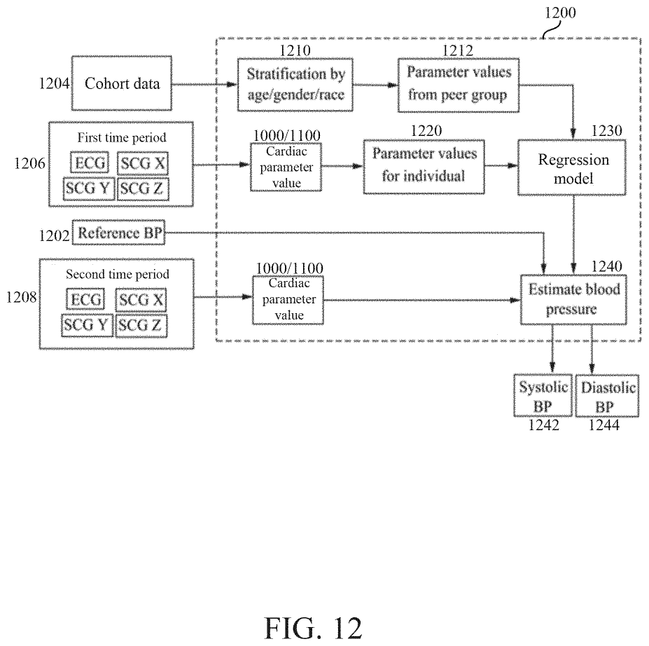

In some variations, the set of values corresponding to at least one mechanical cardiac parameter may be calculated by a computing device as described herein. A blood pressure may then be estimated, for example, by the computing device, using at least one mechanical cardiac parameter. For example, in some variations, the blood pressure may be estimated based on the amplitude. In some variations, the accuracy of a blood pressure estimate may be improved by using a plurality of mechanical cardiac parameters. For example, in some variations, blood pressure may be estimated based on different combinations of mechanical cardiac parameters such as amplitude and maximal L2 norm, and area under the curve, sample entropy, and heart rate. While electrical cardiac data (e.g., ECG data) may be useful in identifying the timepoints of the SCG1 and SCG2 waves in the SCG data, blood pressure may be estimated without directly utilizing the electrical cardiac data. For example, as described in more detail with respect to FIG. 12, a blood pressure estimate may be determined using a regression model based solely on mechanical cardiac parameter values. Put another way, the electrical cardiac data (e.g., ECG data) need not be input to the regression model in order to estimate the blood pressure of a user.

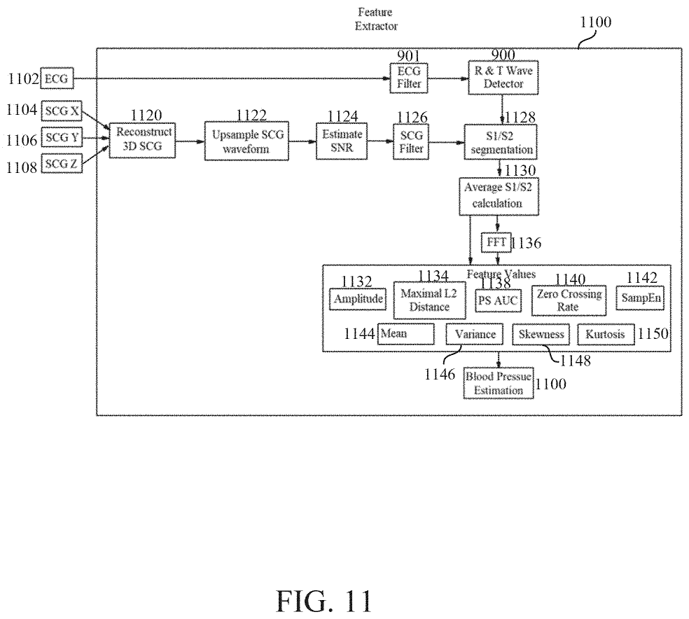

In some variations, blood pressure may be estimated using a combination of mechanical cardiac parameters that may include statistical moments and/or exclude heart rate. FIG. 11 is a flowchart describing a variation of a method of determining mechanical cardiac parameter values (1100). In the variation shown there, four time-dependent waveforms may be utilized to determine the mechanical cardiac parameter values: ECG waveform (1102); X-axis SCG waveform (1104); Y-axis SCG waveform (1106); and Z-axis SCG waveform (1108). ECG and SCG waveforms may be used in the method of determining mechanical cardiac parameter values (1100) for each time period of interest. Each time period of an ECG and SCG waveform may comprise a plurality of heartbeats (e.g., segments) each having an R wave, T wave, SCG wave, and the like. Since the ECG and SCG waveforms are measured synchronously, the R wave timepoint and T wave timepoint from the ECG waveform correspond to the SCG1 wave timepoint and SCG2 wave timepoint as shown in FIGS. 8A and 8B.

The raw ECG waveform (1102) measured using a cardiac monitor may be input to an ECG filter (901) of the cardiac monitor to remove noise and increase the signal-to-noise ratio, as described in detail with respect to FIG. 9. The pre-processed ECG waveform may then be input to R wave and T wave detector (900) and processed as described with respect to FIG. 9. The R wave timepoints and T wave timepoints may be provided as input (1128) as described in more detail herein. Referring again to FIG. 11, the SCG waveforms may be first processed by generating a position invariant, three-dimensional SCG waveform. For example, the three SCG waveforms (1104, 1106, 1108) may be combined into a joint SCG waveform (1120) (SCG_{xyz}). This may be accomplished using equation (3):

$$S C {G}_{x y z} = \sqrt{S C {G}_{x}^{2} + S C {G}_{y}^{2} + S C {G}_{z}^{2}} . \qquad ( 3 )$$

In some variations, the joint SCG waveform SCG_{xyz} may be up-sampled (1122) to about 500 Hertz using linear interpolation. Up-sampling may be optionally performed to reduce computational load on, for example, the cardiac monitor. The signal-to-noise ratio (SNR) of SCG_{xyz} may be calculated (1124), and the joint seismocardiogram waveform SCG_{xyz} may be filtered using an IIR filter (1126). In some variations, portions of the waveform SCG_{xyz} having an SNR below a pre-determined threshold may be excluded from further processing.

The timepoints of the SCG waves (SCG1 (S1) wave, SCG2 (S2) wave) of the SCG_{xyz} waveform may then be determined (1128) (e.g., S1/S2 segmentation). For example, since the ECG and SCG waveforms are measured synchronously, the R wave timepoint and T wave timepoint from the ECG waveform may be used to aid in detecting and/or identifying the timepoints of the SCG1 and SCG2 waves (1128) within a time period.

In some variations, average SCG1 and SCG2 waves (e.g., S1, S2 waves) may be calculated across all segments (1130). Utilizing average SCG1 and SCG2 waves may assist in reducing variance due to motion, speech, and respiration. Additionally, in some instances, cubic spline interpolated rescaling to correct for variability in the SCG amplitude due to heavy respiration may be utilized. Averaging the SCG waves may increase the accuracy of the mechanical cardiac parameter value calculation and subsequent blood pressure estimation. The average SCG1 wave may be used when estimating systolic blood pressure, and the average SCG2 wave may be used when estimating diastolic blood pressure.

A plurality of mechanical cardiac parameters may be calculated from the SCG1 and SCG2 waves (e.g., average SCG1 and average SCG2 waves) (1130). For example, the SCG1 and SCG2 waves may be used to calculate the amplitude (1132), the maximal L2 distance (1134), the area under a section of the power spectral density (PS AUC) (1138), the zero crossing rate (1140), the sample entropy (SampEN) (1142), statistical moments (1144, 1146, 1148, 1150), and the like. Statistical moments may include, for example, mean (1144), variance (1146), skewness (1148), and kurtosis (1150).

In some variations, the zero-crossing rate f₅ (1140) may be the number of times the S wave alternates from positive to negative or vice-versa. The zero-crossing rate (1140) may be calculated individually for each of the filtered SCG waveforms (1104, 1106, 1108). In some variations, it has been empirically observed that higher zero-crossing rates may be indicative of lower blood pressure and may correspond with an S wave experiencing less rapid change.

As used here, the amplitude is the difference between a peak height and a trough height in the average SCG1 wave or SCG2 wave. The maximal L2 distance is the maximum distance between any pair of points on the SCG_{xyz} waveform, and is given by equation (4):

$$Maximal L 2 distance = {max}_{{t}_{1} , {t}_{2}} \sqrt{{\sum }_{i ∈ \{ x , y , z \}} {( SC {G}_{i} [ {t}_{1} ] - S C {G}_{x} [ {t}_{2} ] )}^{2}} \qquad ( 4 )$$

In equation (4), t₁ and t₂ may range over the length of the average SCG1 wave or SCG2 wave. The area under a section of a power spectral density is given by equation (5):

$$PS AUC = \sum {\vert FFT \vert}^{2} \qquad ( 5 )$$

FFT in equation (5) corresponds to a standard Fast Fourier Transform of the SCG waveform (1136), where the sum is over an adjustable frequency band.

In some variations, statistical moments comprising one or more of mean (1144), variance (1146), skewness (1148), and kurtosis (1150) may be calculated from the S wave. In some variations, the higher moments of order q may be normalized by the factor (E((S−E(S))²))^{q/2}. In some variations, it has been empirically observed that the mean (1144) and variance (1146) tend to be the most informative. In general, higher mean and variance relative to the calibration values indicate higher blood pressure, though this may be subject-dependent.

In some variations, the set of values corresponding to at least one mechanical cardiac parameter may be calculated by a computing device as described herein. A blood pressure may then be estimated, for example, by the computing device, using at least one mechanical cardiac parameter. For example, in some variations, the blood pressure may be estimated based on the amplitude. In some variations, the accuracy of a blood pressure estimate may be improved by using a plurality of mechanical cardiac parameters. For example, in some variations, blood pressure may be estimated based on different combinations of mechanical cardiac parameters such as amplitude and maximal L2 norm, and area under the curve, sample entropy, and heart rate. While electrical cardiac data (e.g., ECG data) may be useful in identifying the timepoints of the SCG1 and SCG2 waves in the SCG data, blood pressure may be estimated without directly utilizing the electrical cardiac data. For example, as described in more detail with respect to FIG. 12, a blood pressure estimate may be determined using a regression model based solely on mechanical cardiac parameter values. Put another way, the electrical cardiac data (e.g., ECG data) need not be input to the regression model in order to estimate the blood pressure of a user.

FIG. 12 is a flowchart describing a variation of a method of estimating blood pressure (1200). The method (1200) may comprise receiving a reference blood pressure of a user (1202), cohort data associated with the user (1204), and measured ECG and SCG waveforms corresponding to a first time period (1206) and second time period (1208). The first time period waveforms (1206) may correspond to the waveforms shown in FIG. 8A and the second time period waveforms (1208) may correspond to the waveforms shown in FIG. 8B. In some variations, the first time period waveforms (1206) may correspond to a reference time period with initial measurements obtained after attaching the cardiac monitor to the user and in conjunction with obtaining a reference blood pressure measurement, and the second time period (1208) may be measured continuously or semi-continuously with respect to the first time period. For example, cardiac data (e.g., electrical and mechanical) may be initially measured using the sensors during a reference time period (e.g., first time period) just after the user initially attaches the cardiac monitor to the skin. In some of these variations, blood pressure may be estimated based on the difference between one or more mechanical cardiac parameter values taken at a reference time period and a second time period subsequent the reference time period.

A subset of cohort cardiac data (1204) associated with the user may be retrieved from the cohort cardiac data (1210). For example, demographic categories such as age, gender, race, weight, height, body mass index, and the like may be used to classify a user to an appropriate cohort. A set of values for at least one mechanical cardiac parameter may be retrieved (1212) from the cohort cardiac data associated with the user, and may be input into a regression model (1240) as described in more detail herein.

The regression model (1230) may also receive a set of values for at least one mechanical cardiac parameter. From the mechanical cardiac parameter values generated by the method of determining mechanical cardiac parameter values (1000, 1100) shown in respective FIGS. 10 and 11, a cardiac parameter value for the user β₀ (1220) may be calculated. For example, the cardiac parameter value for the user β₀ may be calculated using equation (6) (1230):

$${β}_{0} = {y}_{0} - {\sum }_{i = 1}^{n} {β}_{i} {x}_{0 , i} \qquad ( 6 )$$

where, y₀ is the reference blood pressure of the user, n is the number of extracted features, x_{0,i} are the mechanical cardiac parameter values calculated based on the first time period waveforms, and βi are the cohort mechanical cardiac parameter values.

The blood pressure estimation method (1240) may receive the reference blood pressure, the cardiac parameter value for the user β₀, and the values of at least one mechanical cardiac parameter of the second time period. For example, the blood pressure estimation may be given by equation (7) and may be formed for systolic blood pressure (1242) and diastolic blood pressure (1244):

$${y}_{1} = {β}_{0} + {\sum }_{i = 1}^{n} {β}_{i} {x}_{1 , i} \qquad ( 7 )$$

where, y₁ is the estimated blood pressure (1240) over the second time period and x_{1,i} are the mechanical cardiac parameter values calculated based on the second time period waveforms. By combining equation (6) and (7), blood pressure estimation may be given by equation (8):

$${y}_{1} = {y}_{0} + {\sum }_{i = 1}^{n} {β}_{i} ( {x}_{i} - {x}_{0} ) \qquad ( 8 )$$

where i is a number of mechanical cardiac parameters, y₁ is the estimated blood pressure of the user, y₀ is the reference blood pressure of the user, βᵢ is an i^{th} cohort mechanical cardiac parameter value, xᵢ is the value of the i^{th} mechanical cardiac parameter of the first time period, and xo is the value of the i^{th} mechanical cardiac parameter of the second time period.

As can be seen from equation (8), the estimated blood pressure may be based on a sum of the changes in the mechanical cardiac parameter values between the first and second time periods for n mechanical cardiac parameters. In some variations, at least one of the mechanical cardiac parameters may be selected from the group consisting of an SCG wave amplitude, a maximal L2 distance, an area under a power spectral density curve, a sample entropy, one or more statistical moments, and an R wave wavelength.

The blood pressure estimate may be calculated using at least one mechanical cardiac parameter. In some variations, mechanical cardiac parameter values may be calculated for predetermined time periods (e.g., continuous, non-continuous) and used to estimate a systolic blood pressure and diastolic blood pressure. These time-dependent estimates may be appended together to form a continuous or semi-continuous user blood pressure tracing. The estimated blood pressure may be output to the user on any accessible computing device. For example, the user may use a graphical user interface (GUI) to view their estimated blood pressure, cardiac data, electrical cardiac parameter data and/or mechanical cardiac parameter data using one or more of a mobile application (e.g., iOS, Android), web browser accessing a secure website, and/or cloud computing solution. The user may register an account through the application and login to access its functionality. The blood pressure and cardiac data may be presented using the GUI in one or more customizable formats that allow a user to gain greater insight to their cardiac health. For example, blood pressure trends may be plotted over time and may include target information, averages, and/or color coding. Cardiac data may be displayed in a table format. Additionally or alternatively, estimated blood pressure and cardiac data may be output using texts, e-mails, and/or other electronic communication methods.

### III. EXAMPLES

As described herein, blood pressure estimates may be derived from electrical and mechanical cardiac data. Examples 1 and 2 below show a set of sphygmomanometer-based measurements compared to blood pressure estimates using the devices, systems, and methods described herein. In particular, reference blood pressures were measured using an oscillometric blood pressure monitor including a blood pressure cuff (e.g., sphygmomanometer). The resting blood pressure was measured for about one minute with the user resting comfortably. Blood pressure was also measured under more strenuous conditions including when the user was solving arithmetic problems, exposed to low intensity stimulus (e.g., listening to relaxing music, viewing a beach scene using a virtual reality headset), and exposed to high intensity stimulus (e.g., experiencing a roller coaster ride using a virtual reality headset).

For examples 1 and 2, the difference between the reference blood pressures and the blood pressure estimates obtained using the method of FIG. 10 were compared against the Association for the Advancement of Medical Instrumentation (AAMI) standards and British Hypertension Society (BHS) standards. AAMI standards require the mean error of the estimate to be less than 5 mmHg and the standard deviation error to be less than 8 mmHg BHS rates blood pressure estimates on a letter grade scale (e.g., A, B, C, D). For activity levels below strenuous exercise, the estimated blood pressure values estimated by the devices, systems, and methods described herein had a standard deviation error of between about 4.75 mmHg and about 8 mmHg.

Similarly, Example 3 below shows blood pressure estimates obtained using the method of FIG. 11 using the devices and systems described herein that meet and also exceed the requirements of ISO/ANSI/AAMI 81060 which provide minimum labeling, performance, and safety requirements for the clinical validation of medical electrical equipment use for the intermittent non-invasive automatic estimation of the arterial blood pressure and is applicable to all sphygmomanometers that sense or display pulsations, flow, or sounds for the estimation, display, or recording of blood pressure. For example, over 100 test subjects were included and blood pressure was measured under low intensity stimulus and high intensity stimulus (e.g., experiencing a roller coaster ride using a virtual reality headset).

### Example 1

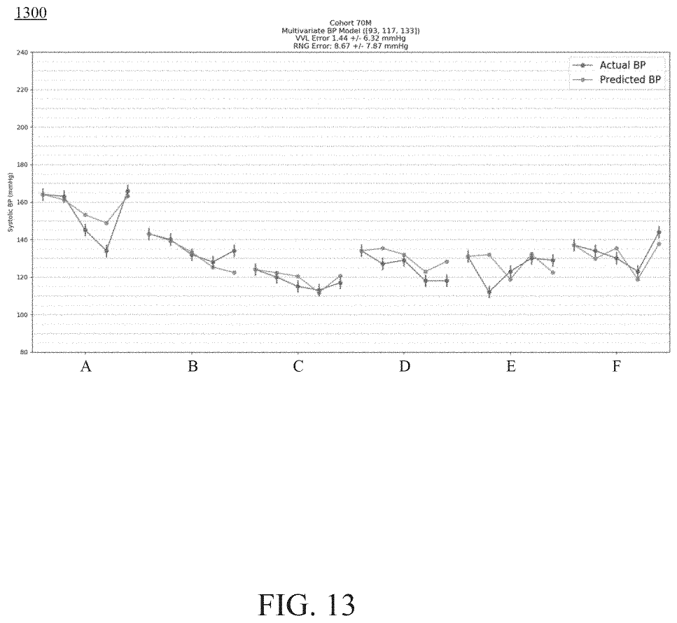

FIG. 13 is a graph comparing measured and estimated blood pressure data for males over 70 years of age. As shown in FIG. 13, estimated blood pressure estimates (“Predicted BP”) show low error relative to sphygmomanometer-based blood pressure measurements (“Actual BP”) for a set of users (A-F). For example, the standard deviation error was about 6.25 mmHg for measurements taken during exercise and about 7.75 mmHg for measurements taken without exercise. The blood pressure estimates of this cohort passed the AAMI standards and earned a BHS A grade.

### Example 2

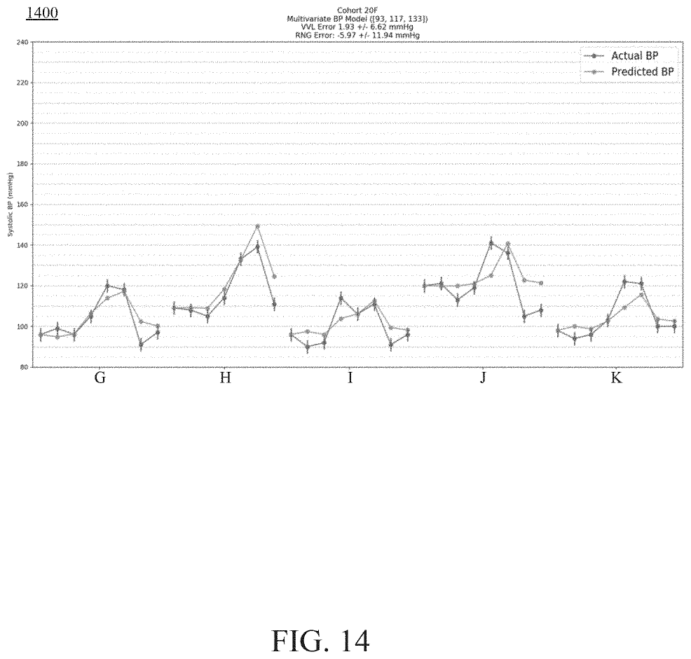

FIG. 14 is a graph comparing measured and estimated blood pressure data for females between 20 and 29 years of age. As shown in FIG. 14, estimated blood pressure estimates (“Predicted BP”) show low error relative to sphygmomanometer-based blood pressure measurements (“Actual BP”) for a set of users (G-K). For example, the standard deviation error was about 6.5 mmHg for measurements taken during exercise and about 5.5 mmHg for measurements taken without exercise. The blood pressure estimates of this cohort passed the AAMI standards and earned a BHS B grade.

### Example 3

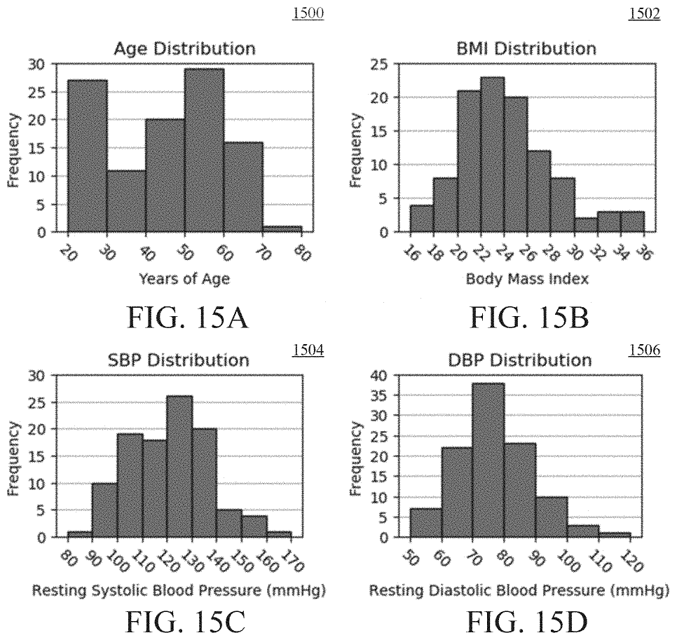

FIGS. 15A-15D are histograms of demographic information for a set of test subjects including age distribution (1500), BMI (1502), resting systolic blood pressure graph (1504), and resting diastolic blood pressure graph (1506). Measurements and analysis were conducted on 104 test subjects, exceeding the minimum of 85 subjects recommended by ISO/ANSI/AAMI 81060. Each subject participated in at least four blood pressure recordings, exceeding the minimum of three recordings per subject recommended by ISO/ANSI/AAMI 81060. The measurements obtained were used to determine the accuracy of the estimated blood pressure values estimated by the devices, systems, and methods described herein. Additionally, measurements were obtained during light exercise on a pedal machine, an arithmetic quiz, a virtual reality roller coaster, and other daily activities, thereby providing more challenging test cases than those suggested by ISO/ANSI/AAMI 81060.

The subject population included a wide range of ages and fitness levels, from underweight (e.g., BMI<18.5) to obese (e.g., BMI>30), and from hypotensive (e.g., systolic blood pressure is<90 mmHg or diastolic blood pressure<60 mmHg) to Stage 3 hypertensive (e.g., systolic blood pressure is between 130 mmHg and 139 mmHg or diastolic blood pressure is between 80 mmHg and 89 mmHg). Additionally, a subset of twelve subjects were taking medication to manage high blood pressure, and their blood pressure recordings were taken over the course of a single day to track the effect of medication on the devices, systems, and methods described herein.

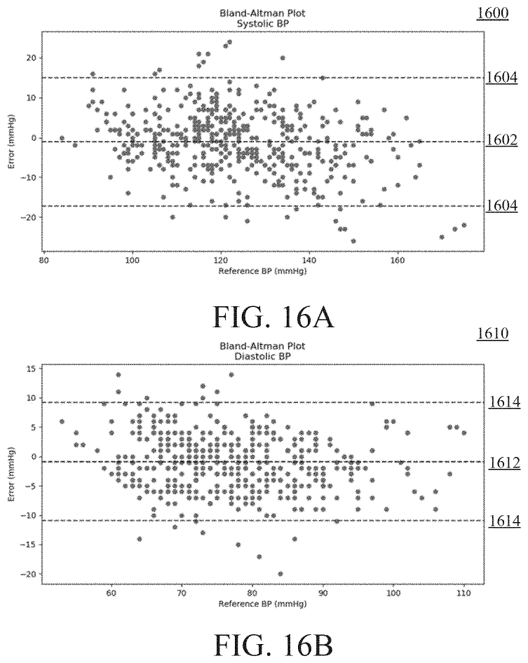

FIG. 16A is a plot (1600) of systolic blood pressure error and FIG. 16B is a plot (1610) of diastolic blood pressure error using a Bland-Altman style plot for evaluating the agreement among two different measurements techniques. Each plot (1600, 1610) comprises a mean error (1602, 1612) and ±2 standard deviations of the error (1604, 1614). The accuracy of the estimated systolic and diastolic blood pressure was evaluated with respect to the thresholds outlined in ISO/ANSI/AAMI 81060 with respect to mean error (criterion 1), standard deviation of error (criterion 2), and intra-subject standard deviation from mean (criterion 3). In particular, the estimated measurements obtained by the devices, systems, and methods described herein were within the mean error of less than ±5 mmHg, standard deviation of error of less than ±8 mmHg, and intra-subject standard deviation from the mean of less than ±8 mmHg, as outlined below in Table 1.

TABLE 1
Statistical Performance

| Criterion 1 (Inter-Subject) | | | | Criterion 2 (Intra-Subject) | |
|---|---|---|---|---|---|
| Mean Error | | Std. Dev. Error | | Std. Dev. Error | |
| (mm Hg) | | (mm Hg) | | (mm Hg) | |
| SYS | DIA | SYS | DIA | SYS | DIA |
| −0.66 | −0.86 | 7.98 | 5.04 | 4.52 | 3.77 |

The threshold for passing criterion 2 depends on the mean error from criterion 1. The larger the mean error, the more difficult it becomes to pass criterion 2, as shown in Table 2 below. In the worst case, when the mean error is ±5 mmHg, the threshold for criterion 2 is 4.79 mmHg.

TABLE 2
Averaged subject data acceptance (criterion 2) in mmHg

| | Maximum permissible standard deviation, [symbol] as function of, [symbol] mmHg | | | | | | | | | |
|---|---|---|---|---|---|---|---|---|---|---|
| [symbol] | 0.0 | 0.1 | 0.2 | 0.3 | 0.4 | 0.5 | 0.6 | 0.7 | 0.8 | 0.9 |
| ±0. | 6.95 | 6.95 | 6.95 | 6.95 | 6.93 | 6.92 | 6.91 | 6.90 | 6.89 | 6.88 |
| ±1. | 6.87 | 6.86 | 6.84 | 6.82 | 6.80 | 6.78 | 6.76 | 6.73 | 6.71 | 6.68 |
| ±2. | 6.65 | 6.62 | 6.58 | 6.55 | 6.51 | 6.47 | 6.43 | 6.39 | 6.34 | 6.30 |
| ±3. | 6.25 | 6.20 | 6.14 | 6.09 | 6.03 | 5.97 | 5.89 | 5.83 | 5.77 | 5.70 |
| ±4. | 5.64 | 5.56 | 5.49 | 5.41 | 5.33 | 5.25 | 5.16 | 5.08 | 5.01 | 4.90 |
| ±5. | 4.79 | — | — | — | — | — | — | — | — | — |

EXAMPLE For mean of [symbol] 4.2 mmHg, the maximum permissible standard deviation is 5.49 mmHg. [symbol]
indicates data missing or illegible when filed

The specific examples and descriptions herein are exemplary in nature and variations may be developed by those skilled in the art based on the material taught herein without departing from the scope of the present invention, which is limited only by the attached claims.

1. A method of estimating blood pressure, comprising:
receiving mechanical cardiac data of a user measured using an accelerometer;
generating, from the mechanical cardiac data, a mechanical cardiac parameter value for a first time period and a second time period; and
estimating the blood pressure of the user based on a change in the mechanical cardiac parameter value between the first and second time periods.

2. The method of claim 1 further comprising receiving a reference blood pressure of a user and cohort cardiac data associated with the user.

3. The method of claim 2, wherein the estimated blood pressure is also based on the reference blood pressure and the cohort cardiac data.

4. The method of claim 1, wherein generating the mechanical cardiac parameter value comprises generating mechanical cardiac parameter values for a plurality of mechanical cardiac parameters, and wherein the estimated blood pressure is based on a sum of the changes in the mechanical cardiac parameter values between the first and second time periods for the plurality of mechanical cardiac parameters.

5. The method of claim 4, wherein at least one of the plurality of mechanical cardiac parameters is selected from the group consisting of an SCG wave amplitude, a maximal L2 norm, an area under a power spectral density curve, a zero crossing rate, a sample entropy, a statistical moment, and an R wave wavelength.

6. The method of claim 1, wherein the mechanical cardiac data comprises an SCG wave.

7. The method of claim 6, wherein the SCG wave comprises an SCG1 wave and an SCG2 wave.

8. The method of claim 6, wherein the mechanical cardiac data comprises a plurality of SCG waves for each of the first time period and the second time period, and wherein generating the mechanical cardiac parameter values comprises generating an average SCG wave for the first time period and the second time period.

9. The method of claim 8, wherein the mechanical cardiac parameter values are derived from the average SCG waves.

10. The method of claim 8, wherein the mechanical cardiac parameter comprises one or more of an SCG wave amplitude, a maximal L2 norm, an area under a power spectral density curve, a zero crossing rate, a sample entropy, a statistical moment, and an R wave wavelength.

11. (canceled)

12. The method of claim 1, further comprising receiving electrical cardiac data measured using an electrode.

13. (canceled)

14. The method of claim 12, wherein the electrical cardiac data is measured as an ECG signal and the mechanical cardiac data is measured as an SCG signal.

15. The method of claim 12, further comprising generating, from the electrical cardiac data, a plurality of electrical cardiac parameter values, wherein the electrical cardiac parameter comprises one or more of a heart rate, an R wave timepoint, and a T wave timepoint.

16. The method of claim 15, wherein generating the plurality of electrical cardiac parameter values comprises generating the R wave timepoint on an ECG waveform using sliding window integration.

17. The method of claim 15, wherein generating the plurality of electrical cardiac parameter values comprises generating the T wave timepoint on an ECG waveform using the R wave timepoint and a derivative of the electrical cardiac data.

18. The method of claim 15, further comprising generating an SCG wave timepoint from the mechanical cardiac data using the R wave and T wave timepoints.

19. The method of claim 1, wherein the mechanical cardiac data comprises first, second, and third seismocardiograph waveforms measured along respective axes, and the method further comprises generating a fourth seismocardiograph waveform comprising the first, second, and third seismocardiograph waveforms.

20. The method of claim 2, wherein estimating the blood pressure is given by:
$${BP}_{est} = {BP}_{ref} + {\sum }_{i = 1}^{n} {β}_{i} ( {x}_{2 , i} - {x}_{1 , i} ) ,$$
where i is an index for a set of n mechanical cardiac parameters, BPₑₛₜ is the estimated blood pressure of the user, BP_{ref} is the reference blood pressure of the user, βᵢ is an i^{th} cohort mechanical cardiac parameter value, x_{1,i} is the first value of the i^{th} mechanical cardiac parameter, and x_{2,i}, is the second value of the i^{th} mechanical cardiac parameter.

21. The method of claim 1, further comprising releasably coupling the accelerometer to skin of the user's left chest over a left ribcage.

22. The method of claim 1, wherein the cohort cardiac data is grouped by one or more of age, gender, race, and body mass index.

23. (canceled)

24. The method of claim 1, wherein the first time period is a reference time period, and wherein the mechanical cardiac data is initially measured using the accelerometer during the reference time period.

25. A method of estimating blood pressure, comprising:
receiving a reference blood pressure of a user, cohort cardiac data associated with the user, and cardiac data of the user at first and second time periods, wherein the cardiac data is measured using an electrode and an accelerometer;
processing the cardiac data to generate first and second values for a mechanical cardiac parameter corresponding to the respective first and second time periods; and
estimating the blood pressure of the user based on the reference blood pressure, the cohort cardiac data, and a change between the first and second values for the mechanical cardiac parameter.

26. The method of claim 25, wherein the mechanical cardiac parameter comprises a plurality of mechanical cardiac parameters, and wherein the estimated blood pressure is based on a sum of the changes in the mechanical cardiac parameter values between the first and second time periods for the plurality of mechanical cardiac parameters.

27. A method of estimating blood pressure, comprising:
receiving a reference blood pressure of a user and cardiac data of the user at first and second time periods, wherein the cardiac data is measured using an electrocardiogram (ECG) sensor and a seismocardiogram (SCG) sensor each attached to the skin of the user's upper left chest;
processing the cardiac data to generate electrical cardiac parameter values corresponding to an R wave timepoint and a T wave timepoint, and to generate first and second values for a mechanical cardiac parameter corresponding to the respective first and second time periods based at least in part on the R wave and T wave timepoints;
receiving cohort cardiac data associated with the user for the mechanical cardiac parameter; and
estimating the blood pressure of the user based on the reference blood pressure, the cohort cardiac data, and a change between the first and second values for the mechanical cardiac parameter.

28. The method of claim 27, wherein the mechanical cardiac parameter comprises a plurality of mechanical cardiac parameters, and wherein the estimated blood pressure is based on a sum of the changes in the mechanical cardiac parameter values between the first and second time periods for the plurality of mechanical cardiac parameters.

29. The method of claim 5, wherein the statistical moment comprises one or more of mean, variance, skewness, and kurtosis.

30. A cardiac monitoring system, comprising:
a cardiac monitor, comprising:
a cardiac sensor comprising an accelerometer, wherein the cardiac sensor is configured to releasably attach to skin over a left ribcage of a user and to measure cardiac data at first and second time periods;
a communication device configured to establish a communication channel; and
a non-transitory processor-readable storage medium configured to be executed by a processor and comprising instructions to:
receive the cardiac data using the communication channel;
generate, from the cardiac data, a mechanical cardiac parameter value for a first time period and a second time period; and
estimate a blood pressure of the user based on a change in the mechanical cardiac parameter values between the first and second time periods.

31.-37. (canceled)

38. A non-transitory processor-readable storage medium configured to be executed by a processor and comprising instructions to:
receive cardiac data of a user for a first time period and a second time period;
retrieve a reference blood pressure of the user and cohort cardiac data associated with the user;
process the cardiac data to generate first and second values for a mechanical cardiac parameter corresponding to the respective first and second time periods; and
estimate a blood pressure of the user based on the reference blood pressure, the cohort cardiac data, and a change between the first and second values for the mechanical cardiac parameter.

39.-40. (canceled)
